(19) United States
(12) Patent Application Publication
(10) Pub. No.: US 2026/0159571 A1
(43) Pub. Date: Jun. 11, 2026

(54) Interleukin-15, PD-1 Antibodies, and Taxanes in Non-Small Cell Lung Cancer

(71) Applicant: ImmunityBio, Inc., San Diego, CA (US)

(72) Inventors: Patrick Soon-Shiong, San Diego, CA (US); Sandip Reddy, San Diego, CA (US)

(21) Appl. No.: 19/407,885

(22) Filed: Dec. 3, 2025

Related U.S. Application Data
(60) Provisional application No. 63/729,079, filed on Dec. 6, 2024.
(60) Provisional application No. 63/795,016, filed on Apr. 25, 2025.

(51) Int. Cl. C07K 14/715; A61K 31/337; A61K 38/00; A61K 39/00; C07K 14/54; C07K 16/28

(52) U.S. Cl. CPC C07K 14/7155; A61K 31/337; C07K 14/5443; C07K 16/2818; A61K 38/00; A61K 2039/54; C07K 2317/76; C07K 2319/00

(57) ABSTRACT

Disclosed herein are methods, compositions and kits for treating non-small cell lung carcinoma (NSCLC) in a human subject, wherein the subject has been previously treated with an immune checkpoint inhibitor, the method comprising administering either an interleukin-15 (IL-15) protein or fusion protein derivative thereof, or an interleukin-15:interleukin-15 receptor subunit alpha (IL-15:IL-15Rα) protein or fusion protein derivative thereof, and a PD-1 inhibitor antibody and a taxane. Further disclosed is a treatment regime for administering either the IL-15 protein or fusion protein derivative thereof, or the IL-15:IL-15Rα protein or fusion protein derivative thereof in combination with administering a PD-1 inhibitor antibody and a taxane.

### CROSS-REFERENCE TO RELATED APPLICATIONS

The application claims the benefit of priority under 35 U.S.C § 119 (e) to U.S. Provisional Patent Application No. 63/729,079, filed Dec. 6, 2024 and to U.S. Provisional Patent Application No. 63/795,016, filed Apr. 25, 2025, the entire disclosures of which are incorporated herein by reference.

### REFERENCE TO SEQUENCE LISTING

This application contains a Sequence Listing submitted electronically as an XML file and is hereby incorporated by reference in its entirety. Said XML file, created on Dec. 3, 2025, is named 8774IB-5.xml and is 1,948 bytes in size.

### BACKGROUND

Lung cancer is the most common cancer and the leading cause of cancer death in both men and women worldwide (Ferlay J., et al. Global Cancer Observatory Cancer Today. 2024). The 2 main types of lung cancer based on biology, therapy, and prognosis are non-small cell lung cancer (NSCLC) and small cell lung cancer (SCLC). There are several types of NSCLC including squamous cell carcinoma, large cell carcinoma, and adenocarcinoma, and NSCLC comprises approximately 80-85% of all lung cancers in 2024 (American Cancer Society, Cancer Facts and Figures 2024. Atlanta: American Cancer Society; 2024). The overall survival rate of individuals with NSCLC between 2012 and 2018 is 28% (American Cancer Society 2024) emphasizing the importance of finding novel therapies for treating individuals with this disease. Furthermore, there is a burden in healthcare costs associated with NSCLC (Zhang X., et al. Health care resource utilization and costs associated with advanced or metastatic nonsmall cell lung cancer in the United States. J Manag Care Spec Pharm. 2022; 28:255-65). As an example, in first-line therapy, the mean cost per patient per year for advanced or metastatic disease between 2010 and 2019 totaled $271,590 for the first-line treatment period and $250, 942 for the study follow-up period (Zhang 2022). Developing novel therapeutics that improve care and long-term outcomes would also aid in easing financial costs both to the individual and to society.

Treatment for NSCLC has advanced over the past decade due to molecularly targeted agents. Yet due to treatment resistance, long-term treatment outcomes have not improved (Acheampong E., et al. Meta-Analysis of Circulating Tumor Cell PD-L1 Expression and the Association with Clinical Outcomes in Non-Small Cell Lung Cancer. Clin Chem. 2024; 70:234-49). Immune checkpoint inhibitors (ICIs) are the current standard of care (SoC) both alone and in combination with chemotherapy in individuals with advanced or metastatic NSCLC. Human ICI antibodies inhibit the programmed death-1 (PD-1) or programmed death-ligand 1 (PD-L1) receptor and thus improve antitumor immunity. Programmed death-1 (PD-1) and PD-L1 expression on circulating tumor cells are the only prognostic markers approved for use in NSCLC. Due to common treatment resistance after first-line therapy, guidelines have been established to address second-line therapies for NSCLC. Treatment guidelines in 2024, from the National Comprehensive Cancer Network in the United States (US) (National Comprehensive Cancer Network Inc. NCCN Guidelines Version 2.2024; Non-Small Cell Lung Cancer. 2024) for individuals with advanced or metastatic disease that have progressed after first-line treatment (with an ECOG performance status score of 0 to 2) include therapy with the immune-checkpoint inhibitors pembrolizumab, nivolumab, or atezolizumab and for category 2B (lower-level evidence), treatment with docetaxel, gemcitabine, ramucirumab/docetaxel, paclitaxel and also pemetrexed for non-squamous NSCLC only.

Pembrolizumab was approved in the US in 2016 and is a humanized immunoglobulin G4 (IgG4) monoclonal antibody that inhibits PD-1 receptors thus improving an antitumor immune response while docetaxel is an antineoplastic agent and is a second-generation chemotherapy of the taxane family. A recent randomized study compared pembrolizumab versus docetaxel as second-line therapy in individuals previously treated with platinum-based chemotherapy and PD-L1 positive advanced NSCLC (Herbst N., et al. Five Year Survival Update From KEYNOTE-010: Pembrolizumab Versus Docetaxel for Previously Treated, Programmed Death-Ligand 1-Positive Advanced NSCLC. J Thorac Oncol. 2021; 16:1718-32). A total of 1034 participants were assessed at 5-years and 893 of 1033 participants in the intent-to-treat population had died. Overall survival (OS) at 5-years was 16.9 months for pembrolizumab and 11.2 months for docetaxel in the PD-L1 tumor proportion score (TPS) ≥50% group and 11.8 months versus 8.4 months in the PD-L1 TPS 1% to 49% group. The most frequently occurring treatment-related adverse events (AEs) in the pembrolizumab group were fatigue, decreased appetite, and rash while common treatment-related AEs in the docetaxel group were alopecia, fatigue, and diarrhea. Immune-mediated AEs and infusion reactions occurred in 23% of participants treated with pembrolizumab and 10% of participants in the docetaxel group. Additionally, a recent study in 62 global centers (Ren et al. KEYNOTE-033: Randomized phase 3 study of pembrolizumab vs docetaxel in previously treated, PD-L1-positive, advanced NSCLC. Int J Cancer. 2023; 153:623-34) further investigated pembrolizumab versus docetaxel as second-line therapy and reported a 1-year OS rate of 12.3 months with pembrolizumab and 10.9 months with docetaxel in the PD-L1 TPS≥50% group and 12.9 months versus 10.6 months in the PD-L1 TPS≥1% group. Frequently reported treatment-related AEs in the pembrolizumab group were increased aspartate aminotransferase, rash, anemia, and hypothyroidism and treatment-related AEs in the docetaxel group included decreased white blood cell count, decreased neutrophil count, leukopenia, nausea, alopecia, and neutropenia. Immune-mediated and infusion reactions occurred in 28.6% of the pembrolizumab group and 6.1% in the docetaxel group.

Nivolumab, a fully humanized IgG4 PD-1 inhibitor, was approved for the treatment of individuals with metastatic squamous-cell and non-squamous cell NSCLC who had disease progression during or after platinum-based chemotherapy based on 2 pivotal trials. In Checkmate-017, 260 participants with advanced squamous-cell NSCLC received treatment with nivolumab (n=131) or docetaxel (n=129), the primary endpoint was OS; secondary endpoints included safety, Investigator-assessed confirmed objective response rate (ORR), progression-free survival (PFS), participant-reported outcomes, and efficacy according to tumor PD-L1 expression (Brahmer J., et al. Nivolumab versus Docetaxel in Advanced Squamous-Cell Non-Small-Cell Lung Cancer. N Engl J Med. 2015; 373:123-35). Nivolumab showed improved efficacy and safety compared to docetaxel. Participants treated with nivolumab showed significantly greater median OS, 9.2 months versus 6.0 months and OS was significantly longer in the nivolumab group. For participants treated with nivolumab the 1-year ORR was 42% versus 24% with docetaxel and the confirmed ORR was 20% with nivolumab versus 9% with docetaxel. Nivolumab monotherapy resulted in fewer grades 3 or 4 treatment-related AEs compared to docetaxel, 7% for nivolumab compared to 55% with docetaxel; 2% of docetaxel patients had grade 5 events. Additionally, nivolumab-treated participants were less likely to discontinue study drug due to treatment related AEs. Frequently reported treatment-related AEs of any grade for nivolumab were fatigue (16% of participants), decreased appetite (11%) and asthenia (10%). Docetaxel participants reported neutropenia (33%), fatigue (33%), alopecia (22%), and nausea (23%). A total of 7% of nivolumab-treated participants reported treatment-related serious AEs of any grade (2% had serious events of grades 3 or 4), compared to the docetaxel group that had a rate of 24% serious AEs at any grade (19% of grade 3 or 4, and 2% grade 5). The most frequently reported treatment-related serious adverse events (SAEs) were hypothyroidism (4% in nivolumab versus 0% in docetaxel), diarrhea (8% versus 20%), increased blood creatinine level (3% versus 2%), and rash (4% versus 6%). A single case each of grade 3 tubulointerstitial nephritis, colitis, and pneumonitis were also reported in the nivolumab group.

Nivolumab showed similar efficacy in metastatic non-squamous NSCLC participants who had progressed during/after platinum-based chemotherapy (Borghaei 2015). Checkmate-057 assessed OS (primary endpoint), safety, and additional secondary endpoints (see above) in 287 nivolumab-treated and 268 docetaxel-treated participants. With a minimum follow-up of 13.2 months, the median OS was 12.2 months in the nivolumab group versus 9.4 months in the docetaxel group. The 1-year ORR was 51% in nivolumab group versus 39% in docetaxel-treated participants (Borghaei H., et al. Five-Year Outcomes From the Randomized, Phase III Trials CheckMate 017 and 057: Nivolumab Versus Docetaxel in Previously Treated Non-Small-Cell Lung Cancer. J Clin Oncol. 2021; 39:723-33). The confirmed ORR was significantly higher in nivolumab participants at 19% versus 12% in the docetaxel group. Treatment-related AEs that were grades 3 and 4 were reported in 10% of nivolumab group compared to 54% for docetaxel participants. Frequently reported treatment-related AEs of any grade for nivolumab were fatigue (16% of participants), nausea (12%), decreased appetite (10%), and asthenia (10%). For docetaxel the most frequently reported treatment-related AE at any grade were neutropenia (31%), fatigue (29%), nausea (26%), and alopecia (25%). Treatment-related serious AEs of any grade occurred in 7% of nivolumab treated participants compared to 20% of docetaxel participants (with 5% versus 18% for grades 3 or 4). The most common any grade treatment-related serious AEs were rash (9% of participants in the nivolumab group versus 3% in the docetaxel group), pruritus (8% versus 1%), erythema (1% versus 4%), diarrhea (8% versus 23%), hypothyroidism (7% versus none), increased alanine aminotransferase (3% versus 1%), increased aspartate aminotransferase level (3% versus 1%), infusion-related reaction (3% versus 3%), and pneumonitis (3% versus <1%). The increased efficacy and lower toxicity for nivolumab monotherapy was later confirmed in Checkmate-171, a phase 2 clinical trial which included metastatic squamous NSCLC participants with broader inclusion criteria (participants could be older and have an Eastern Cooperative Oncology Group (ECOG) score of 0-2 (Felip A., et al. CheckMate 171: A phase 2 trial of nivolumab in patients with previously treated advanced squamous non-small cell lung cancer, including ECOG PS 2 and elderly populations. Eur J Cancer. 2020; 127:160-72), and in daily practice (Knetki-Wroblewska M., et al. Nivolumab for Previously Treated Patients with Non-Small-Cell Lung Cancer-Daily Practice versus Clinical Trials. J Clin Med. 2020; 9). Five-year follow-up data from Checkmate-017 and Checkmate-057 continued to show a survival benefit for nivolumab versus docetaxel (Borghaei H., et al. Five-Year Outcomes From the Randomized, Phase III Trials CheckMate 017 and 057: Nivolumab Versus Docetaxel in Previously Treated Non-Small-Cell Lung Cancer. J Clin Oncol. 2021; 39:723-33). There were 50 nivolumab-treated and 9 docetaxel-treated participants from both trials whose data was available for pooled analysis. The five-year OS was 13.4% for nivolumab and 2.6% for docetaxel. There weren't any participants treated with docetaxel after 2 years, only nivolumab-treated participants were eligible for safety analysis. A total of 67.9% of participants experienced treatment-related AEs, with 10.8% having grades 3 or 4 events. There was no new safety signals observed. Dual therapy has been approved using a treatment regimen of ipilimumab plus nivolumab, both of which are PD-L1 targeted treatments and studies of this regimen in NSCLC are ongoing.

Unmet Medical Need: NSCLC is a difficult to treat disease with limited options for patients after checkpoint inhibitor therapy. It is expected that most individuals will receive PD-1/PD-L1 checkpoint inhibitor therapy in the neoadjuvant, first, and/or second lines of therapy and there are currently no approved therapies specifically for individuals with NSCLC after failure of checkpoint inhibition (the intended use population).

Clinical outcomes were analyzed in a retrospective study of participants with advanced NSCLC that were previously treated with immunotherapy (Li S., et al. Clinical outcomes of atezolizumab versus standard-of care docetaxel with and without ramucirumab in patients with advanced non-small-cell lung cancer who received prior immunotherapy. Front Oncol. 2024; 14:1306311). Participants that were assessed received atezolizumab versus docetaxel with or without ramucirumab as second-line therapy. Participants treated with atezolizumab showed significantly greater OS (17.7 months) versus those participants treated with docetaxel monotherapy (7.7 months) as well as greater OS compared to the docetaxel with ramucirumab group (17.7 months versus 8.9 months). Immunotherapy with atezolizumab did not increase the immune-related AE rate. Additionally, in a systematic review and metanalysis of 9 randomized controlled trials (Wu S., et al. Comparison between the first-line and second-line immunotherapy drugs in the progression-free survival and overall survival in advanced non-small cell lung cancer: a systematic review and meta-analysis of randomized controlled trials. Ann Palliat Med. 2021; 10:1717-26) between first- and second-line immunotherapy (eg, pembrolizumab, nivolumab, atezolizumab) in NSCLC, OS was significantly better in first-line single immunotherapy treatment as compared to second-line single immunotherapy as treatment (HR 0.78, 95% CI: 0.55-1.11, 12=83% versus 0.72, 95% CI: 0.65-0.81, 12=53%). These results suggest that more studies are needed that address combination immunotherapy as a second-line treatment for NSCLC.

The approved agents in second-line NSCLC, docetaxel monotherapy or in combination with ramucirumab and pemetrexed in non-squamous carcinoma, are associated with limited improvement in OS and substantial toxicities. In TAX317, 104 participants were randomized to docetaxel (N=55) versus best supportive care (N=49) resulting in a median OS of 7.5 months for docetaxel compared with 4.6. months for best supportive care (Shepherd 2000). In TAX 320, 104 participants were randomized to docetaxel (N=125) versus vinorelbine or ifosfamide (N=123) resulting in a median OS of 5.7 months for docetaxel compared with 5.6 months for best supportive care (Fossella F V., et al. Randomized phase III trial of docetaxel versus vinorelbine or ifosfamide in patients with advanced non-small-cell lung cancer previously treated with platinum-containing chemotherapy regimens. The TAX 320 Non-Small Cell Lung Cancer Study Group. J Clin Oncol. 2000; 18:2354-62). Together these 2 studies supported the approval of docetaxel in this indication.

Pemetrexed received approval in the second-line NSCLC indication based on a comparison to docetaxel in which 571 participants were randomized and median OS was 8.3 months for pemetrexed and 7.9 months for docetaxel (Hanna N., et al. Randomized phase III trial of pemetrexed versus docetaxel in patients with non-small-cell lung cancer previously treated with chemotherapy. J Clin Oncol. 2004; 22:1589-97). Participants receiving docetaxel compared to pemetrexed were more likely to have grade 3 or 4 neutropenia (40.2% versus 5.3%; P<0.001), febrile neutropenia (12.7% versus 1.9%; P<0.001), and neutropenia with infections (3.3% versus 0.0%; P=0.004).

In the REVEL trial, which led to the approval of ramucirumab in this indication, median OS was 10.5 months for the combination of ramucirumab and docetaxel versus 9.1 months for docetaxel monotherapy (Garon 2014). The authors reported treatment emergent AEs in 613 (98%) of 627 participants in the ramucirumab safety population and 594 (95%) of 618 participants in the control safety population. The most common grade 3 or worse AEs were neutropenia (306 participants [49%] in the ramucirumab group versus 246 [40%] in the control group), febrile neutropenia (100 [16%] versus 62 [10%]), fatigue (88 [14%] versus 65 [10%]), leucopenia (86 [14%] versus 77 [12%]), and hypertension (35 [6%] versus 13 [2%]). The number of deaths from AEs were (31 [5%] versus 35 [6%]) respectively. Given the modest survival times and high toxicity associated with these approved therapies, there exists an urgent unmet medical need for therapies and treatment strategies for individuals in second-line NSCLC.

### SUMMARY

Various embodiments of the invention are described below. However, the invention is not limited to embodiments described in this summary, as inventions described in the description that follows are also expressly encompassed.

One embodiment relates to a method for treating non-small cell lung carcinoma (NSCLC) in a human subject, wherein the subject has been previously treated with an immune checkpoint inhibitor and subsequently experienced disease progression, the method comprising administering to the subject an effective amount of an effective amount of an interleukin-15 (IL-15) protein or fusion protein derivative thereof, or an interleukin-15:interleukin-15 receptor subunit alpha (IL-15:IL-15Rα) protein or fusion protein derivative thereof; an effective amount of a PD-1 inhibitor antibody and an effective amount of a taxane. In one aspect, the IL-15 or variant thereof is in a pharmaceutical composition. In a preferred aspect, the IL-15 or variant thereof is in a pharmaceutical composition formulated for subcutaneous administration. In one aspect, the PD-1 inhibitor antibody is in a pharmaceutical composition. In a preferred aspect, the PD-1 inhibitor antibody is in a pharmaceutical composition formulated for intravenous administration. In one aspect, the taxane is in a pharmaceutical composition. In a preferred aspect, the taxane is in a pharmaceutical composition formulated for intravenous administration.

Another embodiment relates to a composition comprising effective amounts of an interleukin-15 (IL-15) protein or fusion protein derivative thereof, or an interleukin-15:interleukin-15 receptor subunit alpha (IL-15:IL-15Rα) protein or fusion protein derivative thereof; a PD-1 inhibitor antibody; and docetaxel, wherein the composition is formulated for administration to a human subject suffering from NSCLC.

In one aspect of any of the methods, uses or compositions disclosed herein, the NSCLC is advanced or metastatic NSCLC.

In another aspect of any of the methods, uses or compositions disclosed herein, the subject has an actionable genomic alteration (AGA).

In yet another aspect of any of the methods, uses or compositions disclosed herein, the PD-1 inhibitor antibody is an IgG4 antibody.

In still another aspect of any of the methods, uses or compositions disclosed herein, the IL-15:IL-15Rα fusion protein derivative is an IL-15N72D:IL-15RαSu/Fc complex (N-803), wherein the N-803 comprises a dimeric IL-15RαSu/Fc and two IL-15N72D molecules.

In one aspect of any of the methods, uses or compositions disclosed herein the PD-1 inhibitor antibody is selected from the group consisting of tislelizumab, nivolumab, pembrolizumab, MEDI0680 and cemiplimab.

In still another aspect of any of the methods, uses or compositions disclosed herein the PD-1 inhibitor antibody is tislelizumab.

In yet another aspect of any of the methods, uses or compositions disclosed herein, the taxane is docetaxel.

In one aspect of any of the methods, uses or compositions disclosed herein, the IL-15 protein or fusion protein derivative thereof, or the IL-15:IL-15Rα protein or fusion protein derivative thereof, retains binding affinity and agonist activity to interleukin-15 receptor subunits beta gamma (IL-15Rβγ).

In one aspect of any of the methods, uses or compositions disclosed herein, the effective amount of the IL-15 protein or fusion protein derivative thereof, or the IL-15:IL-15Rα protein or fusion protein derivative thereof, is administered subcutaneously to the subject; and the effective amount of the PD-1 inhibitor antibody and the effective amount of the taxane are each administered intravenously to the subject.

In one aspect any of the methods, uses or compositions disclosed herein, the IL-15 protein or fusion protein derivative thereof, or the IL-15:IL-15Rα protein or fusion protein thereof is in a pharmaceutical composition; the PD-1 inhibitor antibody is in a pharmaceutical composition; and the taxane is in a pharmaceutical composition.

In one aspect of any of the methods uses or compositions disclosed herein, the subject is initially administered the IL-15 protein or fusion protein derivative thereof, or the IL-15:IL-15Rα protein or fusion protein derivative thereof, subcutaneously to the subject, followed by administering the PD-1 inhibitor antibody intravenously to the subject, followed by administering the taxane intravenously to the subject, followed by a second cycle of treatment comprising further administering the IL-15 protein or fusion protein derivative thereof, or the IL-15:IL-15Rα protein or fusion protein derivative thereof subcutaneously to the subject, followed by administering the PD-1 inhibitor antibody intravenously to the subject, followed by administering the taxane intravenously to the subject 19 to 23 days after completion of the first cycle.

In still a further aspect, the subject is administered a third cycle of treatment comprising further administering: the IL-15 protein or fusion protein derivative thereof, or the IL-15:IL-15Rα protein or fusion protein derivative thereof, subcutaneously to the subject, followed by administering the PD-1 inhibitor antibody intravenously to the subject 40 to 44 days after the completion of the first cycle. Another embodiment relates to a kit for use in treating NSCLC in a subject, the kit comprising a first vile comprising: a first vile comprising a composition comprising an IL-15 protein or fusion protein derivative thereof, or an IL-15:IL-15Rα protein or fusion protein derivative thereof, wherein the composition is formulated for subcutaneous administration to the subject; a second vile comprising a composition comprising a PD-1 inhibitor antibody, wherein the composition is formulated for intravenous administration to the subject; a third vile comprising a composition comprising a taxane, wherein the composition is formulated for intravenous administration to the subject; and instructions for the use thereof.

In one aspect of the kits disclosed herein, the IL-15:IL-15Rα fusion protein derivative thereof is N-803.

In one aspect of the kits disclosed herein, the PD-1 antibody is tislelizumab.

In one aspect of the kits disclosed herein, the taxane is docetaxel.

Another embodiment relates to a composition comprising: an effective amount of an interleukin-15 (IL-15) protein or fusion protein derivative thereof, or an interleukin-15:interleukin-15 receptor subunit alpha (IL-15:IL-15Rα) protein or fusion protein derivative thereof; an effective amount of the PD-1 inhibitor antibody; and c. an effective amount of the taxane; for use in treating non-small cell lung carcinoma (NSCLC) in a subject, wherein the subject has been previously treated with an immune checkpoint inhibitor and subsequently experienced disease progression.

In one or more of the aspects of the composition for use in treating NSCLC, the NSCLC is advanced or metastatic NSCLC.

In one or more of the aspects of the composition for use in treating NSCLC, the subject has an actionable genomic alteration (AGA).

In one or more of the aspects of the composition for use in treating NSCLC, the PD-1 inhibitor antibody is an IgG4 antibody.

In one or more of the aspects of the composition for use in treating NSCLC, the IL-15:IL-15Rα fusion protein derivative is an IL-15N72D:IL-15RαSu/Fc complex (N-803), wherein the N-803 comprises a dimeric IL-15RαSu/Fc and two IL-15N72D molecules.

In one or more of the aspects of the composition for use in treating NSCLC, the PD-1 inhibitor antibody is selected from the group consisting of tislelizumab, nivolumab, pembrolizumab, MEDI0680 and cemiplimab. In a preferred aspect, the PD-1 inhibitor antibody is tislelizumab.

In one or more of the aspects of the composition for use treating NSCLC, the taxane is docetaxel.

In one or more of the aspects of the composition for use in treating NSCLC, the IL-15 protein or fusion protein derivative thereof, or the IL-15:IL-15Rα protein or fusion protein derivative thereof, retains binding affinity and agonist activity to interleukin-15 receptor subunits beta gamma (IL-15Rβγ).

In one or more of the aspects of the composition for use in treating NSCLC, the effective amount of the IL-15 protein or fusion protein derivative thereof, or the IL-15:IL-15Rα protein or fusion protein derivative thereof, is formulated for subcutaneous administration to the subject; and the effective amount of the PD-1 inhibitor antibody and the effective amount of the taxane are each formulated for intravenous administration to the subject.

In one or more of the aspects of the composition for use in treating NSCLC, the IL-15 protein or fusion protein derivative thereof, or the IL-15:IL-15Rα protein or fusion protein thereof is in a pharmaceutical composition; the PD-1 inhibitor antibody is in a pharmaceutical composition; and the taxane is in a pharmaceutical composition.

In one or more of the aspects of the composition for use in treating NSCLC, the subject is initially administered a first cycle of treatment comprising administering the IL-15 protein or fusion protein derivative thereof, or the IL-15:IL-15Rα protein or fusion protein derivative thereof, subcutaneously to the subject, followed by administering the PD-1 inhibitor antibody intravenously to the subject, followed by administering the taxane intravenously to the subject, followed by a second cycle of treatment comprising further administering the IL-15 protein or fusion protein derivative thereof, or the IL-15:IL-15Rα protein or fusion protein derivative thereof subcutaneously to the subject, followed by administering the PD-1 inhibitor antibody intravenously to the subject, followed by administering the taxane intravenously to the subject 19 to 23 days after completion of the first cycle.

In one or more of the aspects of the composition for use in treating NSCLC, the subject is administered a third cycle of treatment comprising further administering the IL-15 protein or fusion protein derivative thereof, or the IL-15:IL-15Rα protein or fusion protein derivative thereof, subcutaneously to the subject, followed by administering the PD-1 inhibitor antibody intravenously to the subject 40 to 44 days after the completion of the first cycle.

### BRIEF DESCRIPTION OF DRAWING

FIG. 1 depicts study schema.

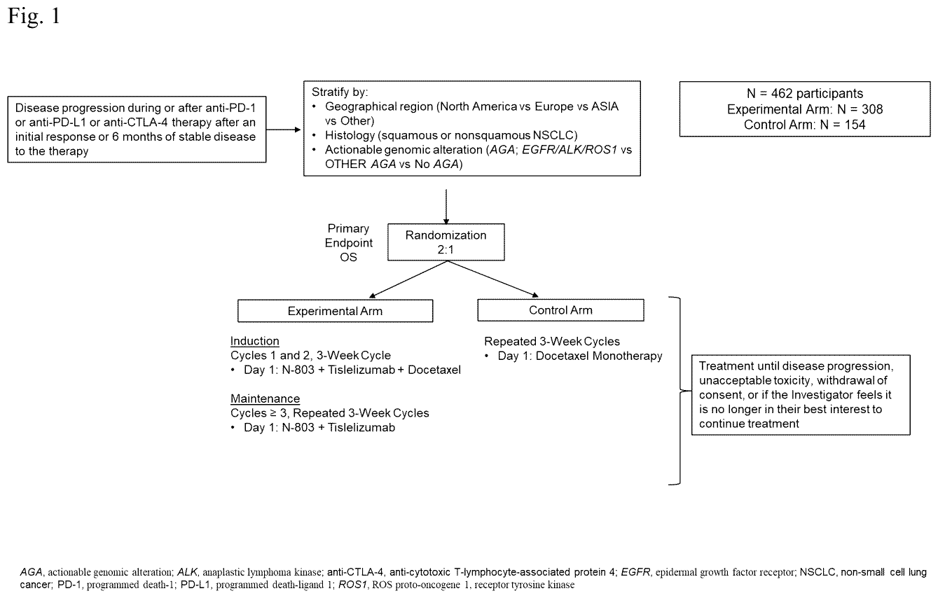

### DETAILED DESCRIPTION

After reading this description it will become apparent to one skilled in the art how to implement the present disclosure in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present disclosure as set forth herein.

Before the present technology is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry are those well-known and commonly used in the art.

Immune checkpoint inhibitors (ICIs) are monoclonal antibodies that bind to immune checkpoint molecules on tumors and tumor antigen-specific T cells. This interaction results in successful immune attack against the tumor by overcoming the “checkpoint” inhibitory signal.

Despite tremendous successes with ICI therapy, most individuals with NSCLC will ultimately have disease progression through a variety of mechanisms which include immune exhaustion, increased PD-1 expression, tumor vascular changes, antigen heterogeneity, expression of secondary checkpoints, and human leukocyte antigen (HLA) downregulation. Thus, immunotherapeutic strategies that enhance the clinical benefits of immune checkpoint blockade in individuals with advanced cancers are clearly needed.

Disclosed herein is a treatment regimen designed to maximize immune cell death (ICD) and augment and maintain the innate and adaptive immune responses against cancer cells. As provided for herein an interleukin-15 (IL-15) protein or fusion protein derivative thereof, or an interleukin-15:interleukin-15 receptor subunit alpha (IL-15:IL-15Rα) protein or fusion protein derivative thereof, including, the IL-15 superagonist, N-803 (also known as “ALT-803”), is used to enhance tumor-specific cytotoxic T-cell responses and to enhance the activity of endogenous NK cells. These proteins and/or fusion proteins are also expected to promote long-term anticancer immune responses through development of memory T cells.

One embodiment disclosed herein relates to a method for treating NSCLC in a human subject, wherein the subject has been previously treated with an immune checkpoint inhibitor and subsequently experienced disease progression. The method comprises administering to the subject an effective amount an IL-15 protein or fusion protein derivative thereof, or an IL-15:IL-15Rα protein or fusion protein derivative thereof; an effective amount of a PD-1 inhibitor antibody and an effective amount of a taxane. In one aspect, the IL-15 protein or fusion protein derivative thereof, or the IL-15:IL-15Rα protein or fusion protein derivative thereof is in a pharmaceutical composition. In one aspect, the PD-1 inhibitor antibody is in a pharmaceutical composition. In one aspect, the taxane is in a pharmaceutical composition.

One embodiment disclosed herein relates to a composition comprising an effective amount an IL-15 protein or fusion protein derivative thereof, or an IL-15:IL-15Rα protein or fusion protein derivative thereof; an effective amount of a PD-1 inhibitor antibody and an effective amount of a taxane, for use in treating NSCLC in a subject, wherein the subject has been previously treated with an immune checkpoint inhibitor and subsequently experienced disease progression.

The two main types of lung cancer based on biology, therapy, and prognosis are non-small cell lung cancer (NSCLC) and small cell lung cancer (SCLC). There are several types of NSCLC including squamous cell carcinoma, large cell carcinoma, and adenocarcinoma, and NSCLC comprises approximately 80-85% of all lung cancers in 2024 (American Cancer Society 2024). In one aspect of any of the methods, uses or compositions disclosed herein, the NSCLC is advanced or metastatic NSCLC.

In one or more aspects of any of the methods, uses or compositions disclosed herein, the subject has an actionable genomic alteration (AGA). A genomic alteration can be considered “actionable” if it: predicts therapy response (sensitivity or resistance), has demonstrated the ability to establish diagnosis or influence prognosis, affects the function of a cancer-related gene and can be targeted directly or indirectly with approved or investigational therapies, is a specific eligibility criterion for enrollment onto genotype-selected trials, is a germline alteration that predicts drug metabolism and/or adverse effects, is a germline alteration that predicts future risk of cancer or other diseases (usually considered more “actionable” if prevention or screening with early treatment is feasible) (Meric-Bernstam F et al. A decision support framework for genomically informed investigational cancer therapy. J Natl Cancer Inst., April 11; 107(7), pg 1-9 2015).

### Interleukin-15 (IL-15)

An “interleukin-15 protein” or “IL-15” as referred to herein includes any of the recombinant or naturally-occurring forms of the interleukin-15 (IL-15) protein, or derivatives (including variants and/or homologs) thereof, or IL-15 fusion protein derivatives thereof, that maintain IL-15 protein activity (e.g. within at least 50%, 80%, 90%, 95%, 96%, 97%, 98%, 99% or 100% activity compared to IL-15 protein). In embodiments, the IL-15 derivatives or the IL-15 fusion protein derivatives at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-15 protein or an IL-15 portion of a fusion protein comprising IL-15. In embodiments, the IL-15 protein or IL-15 portion of a fusion protein is substantially identical to the protein identified by the UniProt reference number P40933 or a derivative having substantial identity thereto. In one aspect, the IL-15 protein is a fusion protein or derivative thereof.

An “interleukin-15 receptor subunit alpha protein” or “IL-15Rα” as referred to herein includes any of the recombinant or naturally-occurring forms of the interleukin-15 receptor subunit alpha (IL-15Rα) protein or derivatives (including variants and/or homologs) thereof that maintain IL-15Rα protein activity (e.g. within at least 50%, 80%, 90%, 95%, 96%, 97%, 98%, 99% or 100% activity compared to IL-15Rα protein). In embodiments, the derivatives at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-15Rα protein or an IL-15Rx portion of a fusion protein comprising IL-15Rα. In embodiments, the IL-15Rα protein is substantially identical to the protein identified by the UniProt reference number Q13261 or a derivative having substantial identity thereto.

As used herein, “domain” refers to a conserved portion of a protein that functions and exists independently of the rest of the protein sequence. A domain may form a stable, three-dimensional structure that exists as a functional unit independent of the remaining protein. For example, the IL-15RαSu domain is the portion of IL-15Rα that retains the IL-15 binding activity.

As used herein, “IL-15 domain” refers to a polypeptide comprising at least a portion of a sequence of the IL-15 protein. In embodiments, the IL-15 domain comprises at least a portion of the sequence of the IL-15 protein and includes one or more amino acid substitutions or deletions within the amino acid sequence of the IL-15 protein. In embodiments, the IL-15 domain is an IL-15 derivative that comprises a different amino acid sequence compared to the IL-15 protein. In embodiments, the IL-15 domain binds the IL-15Rα protein or a fragment thereof. In embodiments, the IL-15 domain is bound to the IL-15Rα protein or a fragment thereof. In embodiments, the sequence of the IL-15 domain has at least one amino acid change, e.g. substitution or deletion, compared to the IL-15 protein. In embodiments, the amino acid substitutions/deletions are in the portions of IL-15 that interact with IL-15Rβ and/or γC. In embodiments, the amino acid substitutions/deletions do not affect binding to the IL-15Rα polypeptide or the ability to produce the IL-15 domain. In embodiments, the amino acid substitutions/deletions increase or decrease binding affinity of IL-15 to the IL-15Rα domain, the IL-15Rβ domain, or the IL-15Rγ domain. In embodiments, amino acid substitutions can be conservative or non-conservative changes and insertions of additional amino acids compared to the IL-15 protein. In embodiments, the IL-15 domain comprises one or more than one amino acid substitutions/deletions at position 6, 8, 10, 61, 65, 72, 92, 101, 104, 105, 108, 109, 111, or 112 of the IL-15 protein sequence. In embodiments, the IL-15 domain comprises an N72D substitution of the IL-15 protein sequence.

The term “sushi domain” as used herein refers to a common motif in proteins comprising a beta-sandwich arrangement. Sushi domains are common in protein-protein interactions, and typically include four cysteines forming two disulfide bonds in a 1-3 and 2-4 pattern. For example, the region of IL-15Rα that binds IL-15 includes a sushi domain.

In embodiments, the IL-15Rα sushi domain includes the amino acid sequence comprising the sequence of SEQ ID NO:1. In embodiments, the derivatives have at least 90%, 95%, 96%, 97%, 98%, 99% or 100% amino acid sequence identity across the whole sequence or a portion of the sequence (e.g. a 50, 100, 150 or 200 continuous amino acid portion) compared to the sequence of SEQ ID NO:1. In embodiments, the IL-15Rα sushi domain associates with the IL-15 protein. In embodiments, the IL-15Rα sushi domain associates with the IL-15 domain.

(SEQ ID NO: 1)
Ile Thr Cys Pro Pro Pro Met Ser Val Glu His Ala
Asp Ile Trp Val Lys Ser Tyr Ser Leu Tyr Ser Arg
Glu Arg Tyr Ile Cys Asn Ser Gly Phe Lys Arg Lys
Ala Gly Thr Ser Ser Leu Thr Glu Cys Val Leu Asn
Lys Ala Thr Asn Val Ala His Trp Thr Thr Pro Ser
Leu Lys Cys Ile Arg

In embodiments, IL-15 protein or fusion protein derivative thereof or at least one member of an IL-15:IL-15Rα binding pair (covalently or non-covalently linked) is fused to a protein such as Ig or Tissue Factor, wherein the fusion confers pharmacokinetic stability on the IL-15 or IL-15:IL-15Rα. The IL-15 protein or fusion protein derivative thereof, or the IL-15:IL-15Rα protein or fusion protein derivative thereof, retains binding affinity and agonist activity to interleukin-15 receptor subunits beta gamma (IL-15Rβγ). In embodiments, the pharmaceutical composition may comprise a PD-1 inhibitor antibody, a taxane, and a novel IL-15Rγβ agonist such as an agonist antibody or a small molecule agonist.

### N-803

N-803 (nogapendekin alfa inbakicept; also known as ALT-803; recombinant human super agonist interleukin-15 (IL-15) complex [also known as IL 15N72D:IL-15RαSu/IgG1 Fc complex])) is a soluble complex consisting of two protein subunits of a human IL-15 variant (nogapendekin alfa) bound with high affinity to a dimeric human IL-15Rα sushi domain/human IgG1 Fc fusion protein (inbakicept) (Han K P, Zhu X, Liu B, et al. IL-15:IL-15 receptor alpha superagonist complex: high-level coexpression in recombinant mammalian cells, purification and characterization. Cytokine. 2011; 56:804-10; Zhu X, Marcus W D, Xu W, et al. Novel human interleukin-15 agonists. J Immunol. 2009; 183:3598-607). The IL-15 derivative is a 114-amino acid polypeptide comprising the mature human IL 15 cytokine sequence with an Asn-to-Asp substitution at position 72 of helix C (N72D) (Zhu X, Marcus W D, Xu W, et al. Novel human interleukin-15 agonists. J Immunol. 2009; 183:3598-607). The human IL-15Rα sushi domain/human IgG1 Fc fusion protein comprises the sushi domain of the human IL-15 receptor a subunit (IL-15Rα) (amino acids 1-65 of the mature human IL-15Rα protein) linked with the human IgG1 CH2-CH3 region containing the Fc domain (232 amino acids). Aside from the N72D substitution, all the protein sequences are human. N-803 acts as a growth and activation factor for natural killer (NK) cells, and effector and memory T cells.

In any of the methods, uses or compositions disclosed herein, the IL-15:IL-15Rα fusion protein derivative is an IL-15N72D:IL-15RαSu/Fc complex (N-803), wherein the N-803 comprises a dimeric IL-15RαSu/Fc and two IL-15N72D molecules.

In one aspect, the IL-15 protein or fusion protein derivative thereof, or the IL-15:IL-15Rα protein or fusion protein derivative thereof such as N-803, is administered systemically, for example, formulated in a pharmaceutically-acceptable buffer such as physiological saline. Preferable routes of administration include, for example, subcutaneous (SC), intravenous (IV), intraperitoneal, intramuscular, or intradermal injections that provide continuous, sustained levels of the composition in the patient. Treatment of human patients or other animals is carried out using a therapeutically effective amount of a therapeutic identified herein in a physiologically-acceptable carrier. Suitable carriers and their formulation are described, for example, in Remington's Pharmaceutical Sciences by E. W. Martin. In a preferred aspect, the administration route is via subcutaneous administration.

Compositions comprising IL-15 protein or fusion protein derivative thereof, or the IL-15:IL-15Rα protein or fusion protein derivative thereof for parenteral use may be provided in unit dosage forms (e.g., in single-dose ampoules, syringes or bags), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Furthermore, the composition may include suspending, solubilizing, stabilizing, pH-adjusting agents, tonicity adjusting agents, and/or dispersing, agents.

As indicated above, the IL-15 protein or fusion protein derivative thereof, or the IL-15:IL-15Rα protein or fusion protein derivative thereof and/or a composition comprising IL-15 protein or fusion protein derivative thereof, or the IL-15:IL-15Rα protein or fusion protein derivative thereof may be in a form suitable for sterile injection.

Exemplary effective weight-based doses via SC administration of IL-15 protein or fusion protein derivative thereof, or the IL-15:IL-15Rα protein or fusion protein derivative thereof (including N-803) include between 0.1 μg/kg and 50 μg/kg body weight, e.g., 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, or 50 μg/kg. Further, exemplary flat doses (SC administration) include between 0.4 mg and 5 mg (e.g., 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5., 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9 or 5.0 mg) is also acceptable, with a flat dose of 1.2 mg being preferred. As an example, a weight-based dose of 15 μg/kg SC has been safely used for numerous clinical trials of N-803 in advanced cancers, although a flat dose of 1.2 mg SC is also acceptable depending on study parameters. This dose was determined via conversion of the weight-based dose of 15 μg/kg using the average body weight of adults living in the US. Still further, doses administered intravesically include between 300 μg to 500 μg (e.g., 300, 305, 310, 315, 320, 325, 330, 335, 340, 345, 350, 355, 360, 365, 370, 375, 380, 385, 390, 395, 400, 405, 410, 415. 420, 425, 430, 435, 440, 445, 450, 455, 460, 465, 470, 475, 480, 485, 490 or 500 μg.

### N-803 Pharmacology Studies

Nonclinical pharmacology studies provide evidence supporting the induction of trained innate immune memory as the mechanism of action of N-803. This first-in-class IL-15 superagonist N-803 binds to IL-2 receptor β and γc subunits on the surface of CD4+ and CD8+ T cells and NK cells, with higher affinity than wildtype IL-15. N-803 binding to its receptor results in proliferation and activation of CD4+, CD8+ T cells and NK cells, but unlike IL-2, does not induce proliferation of immunosuppressive regulatory T (Treg) cells. N-803 activates CD8+CD44high memory T cells to proliferate and secrete interferon (Luo)-γ. IFN-γ plays a role in augmenting the cytotoxicity of these cells against tumors.

Systemic administration of N-803 resulted in antigen independent activation of NK cells and CD8+ T cells. This activation resulted in enhanced antitumor activity in multiple myeloma, lymphoma, melanoma, lung cancer, and bladder cancer models in rodents. In addition, N-803 has demonstrated promising in vivo and in vitro activity against viruses including human immunodeficiency virus (HIV) in nonclinical models. Further, N-803 administration was tolerated in the various models tested and long term and short-term studies showed that N-803 administration did not induce massive cytokine release (i.e., cytokine storm).

### N-803 Pharmacokinetic Studies

Nonclinical pharmacokinetic (PK) and distribution studies were performed with N-803 using systemic routes of administration (intravenous [IV] and subcutaneous [SC]). Single dose PK studies were performed in mice (3 studies) and rats (1 study), and a repeat dose PK study was conducted in cynomolgus monkeys. Additionally, a distribution study was conducted to evaluate localization in target-expressing mouse cells and organ distribution following IV administration in mice. The results from the three studies in mice and studies each in rats and monkeys suggest that the half-life (t½) of N-803, when administered systemically, is less than 24 hours. In the mouse studies, the high maximum serum concentration (Cmax) and area under the concentration time-curve over the time interval from 0 extrapolated to infinity (AUC0-∞) were lower with SC administration versus IV administration. A distribution study in mice following IV administration showed N-803 uptake in the kidneys, liver, lungs, spleen, and lymph nodes, with persistence in the lymph nodes for at least 70 hours following systemic administration. The study also demonstrates that N-803 may be primarily cleared via the hepatobiliary pathway.

### N-803 Toxicology Studies

Toxicology studies in mice, rats, and monkeys demonstrated the expected immune stimulatory effects. There are no off-target toxicities which preclude the use of N-803 in clinical studies. In general, the incidence and/or severity of toxicities was reversed by the end of the recovery period. N-803 related hematology parameter changes were observed in the studies in mice and monkeys. These changes are consistent with an N-803-mediated shift from erythropoiesis to leukocyte production, which is expected based on the immunostimulatory mechanism of action of N-803. Data from IV or SC routes of administration suggests that there are no local tolerance concerns. In the Good Laboratory Practice (GLP) rat (20328204) and monkey repeat-dose toxicology studies (YLP-1203), no abnormal findings were observed in the cardiovascular, central nervous, and respiratory systems. Overall, the toxicology data demonstrates that N-803 is well-tolerated even with systemic routes of administration.

### N-803 Subcutaneous Dosing for Advanced Cancers

Intravenous (IV) and SC administration of N-803 were compared in early phase 1 studies in solid and hematologic tumors (QUILT-3.002 [NCT02384954], QUILT-3.003 (NCT01946789), QUILT-3.004 (NCT01885897), and QUILT-3.005 [NCT02099539]). The decision to discontinue IV administration was based on the Cmax observed via this route, which was associated with increased serum cytokine levels and high fever, rigors, tachycardia, and relative hypotension compared to SC administration (Romee R., et al. First-in-human phase 1 clinical study of the IL-15 superagonist complex ALT-803 to treat relapse after transplantation. Blood. 2018; 131:2515-27). SC administration of N-803 results in an approximately 100-fold lower Cmax than IV and allows N-803 serum concentration to be maintained up to 96 hours post dose with still detectable levels at 7 days (while IV N-803 declined below detection 48-96 hours post administration) (Margolin K., et al. Phase I Trial of ALT-803, A Novel Recombinant IL15 Complex, in Patients with Advanced Solid Tumors. Clin Cancer Res. 2018; 24:5552-61; Romee R., et al. 2018).

SC administration resulted in attenuation of AEs as compared to IV administration. Participants receiving N-803 via SC administration commonly reported injection site reaction, mild fever, hypoalbuminemia, and chills, while those receiving IV N-803 commonly reported higher fever, rigors, fatigue, nausea, and hypertension. The common localized skin rash observed with SC administration of N-803 could be managed with topical steroids when necessary.

A PK study in healthy volunteers (QUILT-1.004 [NCT03381586]) provided additional evidence of the favorable safety and tolerability profile of SC-administered N-803. Serum levels of N-803 remained elevated over a relatively prolonged period after a single administration of the drug; the apparent mean t½ of the complex was 20-20.7 hours.

In addition to its favorable tolerability and PK profile, SC administration of N-803 is associated with equivalent and possibly enhanced efficacy and biological activity. In QUILT-3.004 (hematologic malignancies), SC administration of N-803 was associated with a greater increase in circulating NK cells than IV administration (Romee R., et al. 2018). In the phase 1 portion of QUILT-3.002 (relapsed or refractory indolent non-Hodgkin lymphoma [iNHL]), the combination of rituximab with N-803 led to objective responses in 67% (8 out of 12) of SC-treated participants and 44% (4 out of 9) of IV-treated participants (Foltz J A., et al. Phase 1 trial of N-803, an IL-15 receptor agonist, with rituximab in patients with indolent non-Hodgkin lymphoma. Clinical Cancer Research. 2021; doi: 10.1158/1078-0432.CCR-20-4575). In the phase 2 portion of QUILT-3.002, the ORR was 63% (95% CI: 40.6, 81.2). Dosing of N-803 for HIV+ participants (6 μg/kg SC) was shown to be safe and well tolerated and to induce biological activity (QUILT-1.001 [NCT02191098]).

In total, these observations show that SC administration is well tolerated, yields a lower Cmax yet more sustained serum levels of N-803 over time, and has equivalent or greater efficacy and biological activity as compared to IV administration.

### N-803 as a Single Agent in Advance Cancers.

N-803 has demonstrated anticancer activity as a monotherapy in patients with hematologic malignancies who relapsed after allogeneic hematopoietic cell transplantation, where clinical benefit was observed in 19% (1 complete response [CR], 1 partial response [PR], 3 stable disease [SD]) of evaluable participants in QUILT-3.004 (Romee 2018). SD was the best response in phase 1 monotherapy studies in patients with solid tumors (QUILT-3.003) (Margolin 2018) and multiple myeloma (QUILT-3.005). N-803 induced proliferation and/or activation of NK cells and CD8+ T cells in both QUILT-3.003 and QUILT-3.004.

N-803 in Combination with Checkpoint Inhibitors and Targeted Therapies in Advance Cancers.

N-803 has demonstrated efficacy and safety when administered in combination with other therapies. In completed studies, N-803 was administered with nivolumab in patients with NSCLC (QUILT-2.002) and with rituximab in patients with relapsed and refractory iNHL (QUILT-3.002). In QUILT-2.002, a post-hoc analysis showed that N-803+nivolumab treatment resulted in objective responses (all PRs) in 6 of 21 participants with NSCLC (Wrangle J M., et al. ALT-803, an IL-15 Superagonist, in Combination with Nivolumab in Metastatic Non-Small Cell Lung Cancer, a non-randomized, open-label, phase 1b trial. Lancet Oncol. 2018; 19(5): 694-704). Notably, 3 of these participants had previously received anti-PD-1 therapy, suggesting a role for N-803 in re-inducing anticancer activity of checkpoint inhibitors in patients whose disease has relapsed after previous treatment with anti-PD-1 monotherapy. Importantly, N-803 did not increase the risk of immune-mediated AEs associated with nivolumab, with only one episode of grade 2 pneumonitis observed on trial that resolved without steroid treatment.

The QUILT-3.002 study in patients with iNHL demonstrated that N-803 could be safely administered with rituximab (Foltz J A., et al. 2021). In the phase 1 portion of the study, there were 3 SAEs reported and all were unrelated to N-803. There were no AEs leading to death and only 1 AE leading to discontinuation (unrelated instance of grade 2 squamous cell carcinoma). In the phase 2 portion of the study, there were no deaths, 3 treatment-related SAEs, and no treatment-related treatment-emergent adverse events (TEAEs) leading to treatment discontinuation. In addition, treatment-related TEAEs occurring in >30% of participants in this study were similar to the most common adverse reactions seen for single-agent rituximab (i.e., mostly related to either infusion or injection site reactions).

In phase 1, participants receiving N-803 SC had ORR of 67% and a CR rate of 58%, while participants receiving N-803 IV had an ORR of 44% and a CR rate of 33%. Overall ORR in phase 2 was 63%.

In ongoing studies, combination therapy with N-803 has been well-tolerated. An interim analysis of QUILT-3.055, in which patients with advanced cancers were treated with N-803 and immune checkpoint inhibitors, showed that the most common N-803-related AEs were injection site reaction, chills, fatigue, and fever. Similarly, in QUILT-88, in which patients with advanced or metastatic pancreatic cancer were administered N-803, aldoxorubicin and cell therapy, the most frequently reported treatment-related (i.e., related to any study drug) AEs were chills, fever, anemia, injection site reaction, and fatigue.

### Taxanes

Taxanes are a class of chemotherapy drugs/antineoplastic agents that treat cancer. Different classes of chemotherapy drugs use different processes to kill cancer cells and prevent tumor growth. Taxanes interfere with cell division or mitosis and thus are considered “mitotic inhibitors”. Current taxanes include paclitaxel used for treating advanced ovarian cancer, metastatic breast cancer, Kaposi sarcoma and NSCLC; docetaxel used for treating breast cancer, NSCLC prostate cancer, gastric adenocarcinoma, and head and neck cancer; cabazitaxel for treating metastatic prostate cancer (my.clevelandclinic.org/health/drugs/24914-taxane), and Abraxane for treating breast, pancreatic, and non-small cell lung cancer.

Exemplary effective doses or amounts of a taxane is between 25 to 125 mg/m² (e.g., 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124 or 125 mg/m²) administered intravenously. A preferred effective amount is 75 mg/m².

A preferred taxane is docetaxel. It is prepared by semi-synthesis beginning with a precursor extracted from the renewable needle biomass of yew plants. The preferred dose/amount of docetaxel is 75 mg/m² administered intravenously over 1 hour every 3 weeks. Docetaxel is available as an injectable solution of 20 mg per 2 mL single-dose vial, 80 mg per 8 mL multiple dose vial or 160 mg per 16 mL multiple dose vial (Alembic Pharmaceuticals Limited).

### Immune Checkpoint Inhibitors

As noted above, immune checkpoint inhibitors (ICIs) are monoclonal antibodies that bind to immune checkpoint molecules on tumors and tumor antigen-specific T cells. This interaction results in successful immune attack against the tumor by overcoming the “checkpoint” inhibitory signal. Such ICIs include antibodies against CTLA-4, PD-1, PD-L1, PD-L2, B7-H3, B7-H4, LAG-3, BTLA, TIM-3, VISTA, IDO, A2aR, HVEM, KIRs, NKG2A, NKG2D, CEACAM-1, 2B4, CD200R, and their ligands and other targets described herein may be capable of promoting immune responses by inhibiting immune suppressive signals. Additionally, preferred antibodies of the invention may include ipilimumab and tremelimumab (anti-CTLA4) as well as PD-1 inhibitor/blocking antibodies (e.g., tislelizumab, nivolumab, pembrolizumab, pidilizumab, cemiplimab and MEDI0680) and other antibodies such as TSR-042, ANB011, AMP-514 and AMP-224 (a ligand-Fc fusion) (anti-PD1), MPDL3280A, MEDI4736, and BMS-9365569 (anti-PDL1), MEDI6469 (anti-OX40 agonist), BMS-986016, IMP701, IMP731, and IMP321 (anti-LAG3). A preferred ICI is a PD-1 inhibitor antibody, including but not limited to tislelizumab, nivolumab, pembrolizumab, MEDI0680 and cemiplimab.

Exemplary effective doses or amounts of a PD-1 inhibitor antibody is between 100 mg and 300 mg (e.g., 105, 110, 115, 120, 125, 130, 135, 135, 140, 145, 150, 155, 160, 165, 170, 175, 180, 185, 190, 195 200, 205, 210, 215, 220, 225, 230, 235, 240, 245, 250, 255, 260, 265, 270, 275, 280, 285, 290, 295 or 300 mg) with a preferred amount being 200 mg to 240 mg.

Tislelizumab (also known as BGB-A317) is a humanized IgG4-variant mAb against human PD-1 that is being developed for treatment of human malignancies in multiple organs and tissues and is a preferred PD-1 inhibitor antibody. The preferred dose/amount of tislelizumab is 200 mg. Preferably this amount is administered as an IV infusion. Tislelizumab is indicated for the treatment of adult patients with unresectable or metastatic esophageal squamous cell carcinoma after prior systemic chemotherapy that did not include a PD-(L) 1 inhibitor.

Tislelizumab binds to the extracellular domain of human PD-1 with high specificity and affinity (dissociation constant=0.15 nM), as demonstrated by receptor binding assays based on surface plasmon resonance. Tislelizumab competitively blocks the binding of both PD-L1 and program death ligand-2 (PD-L2) to PD-1. Robust antitumor activity of tislelizumab was also observed in human PD-1 transgenic mice transplanted with B16/F10 mouse melanoma cells overexpressing murine granulocyte-macrophage colony-stimulating factor.

The IgG4-variant antibody has demonstrated very low binding to gamma fragment crystallizable region receptor (FcγR) RI, RIIIA, and C1q by in vitro assays, suggesting low or no antibody-dependent cellular phagocytosis, antibody-dependent cellular cytotoxicity, and complement-dependent cytotoxicity effects in humans.

Tislelizumab binds to the PD-1 of cynomolgus monkey and human with a similar affinity but does not bind to mouse PD-1. Cynomolgus monkey is therefore considered the relevant animal species for nonclinical studies. In single-dose and 13-week repeated-dose toxicology studies in cynomolgus monkeys, no specific concerns were identified in vital organ systems, including the cardiovascular system, central nervous system, and respiratory system.

A PK study was conducted in cynomolgus monkeys at single doses of 3, 10, or 30 mg/kg or at a repeated dose of 10 mg/kg weekly for 5 doses via intravenous infusion. The systemic exposure appeared to increase dose proportionally without sex difference or accumulation. After single-dose administration of 3, 10, or 30 mg/kg, the half-life (t_{1/2}) ranged from 74 to 183 hours; maximum serum concentration (Cₘₐₓ) ranged from 90 to 999 μg/mL; area under the curve (AUC₀₋₁₀₀₈ₕ) ranged from 12,322 to 163,755 h·μg/mL; and volume of distribution (Va) ranged from 22 to 52 mL/kg.

In single-dose toxicity studies in both mice and cynomolgus monkeys, no mortality or apparent toxicity was noted at a single dose up to 100 mg/kg. In a repeated-dose study in cynomolgus monkeys, no apparent toxicity was noted after intravenous infusion of tislelizumab at 3, 10, or 30 mg/kg once every 2 weeks for 13 weeks (7 dose administrations). Thus, with sustained systemic drug exposure, the efficacy of the drug would not be impacted. The no-observed-adverse-effect level was considered to be 30 mg/kg in this study.

A total of 5 studies have examined the efficacy of tislelizumab in NSCLC suggesting that the agent has biological activity in advanced lung cancer. These studies are BGB-A317-206 (NCT03432598; CTR20170361), BGB-A317-303 (NCT03358875; 2018-000245-39; CTR20171112), BGB-A317-304 (NCT03663205; CTR20180032), BGB-A317-307 (NCT03594747; CTR20180292) and BGB-A317-315 (NCT04379635; CTR20200821)

Nivolumab which is a PD-1 blocking/inhibitor antibody is also preferred.

Nivolumab is an IgG4 kappa immunoglobulin that has a calculated molecular mass of 146 kDa. It is expressed in a recombinant Chinese Hamster Ovary (CHO) cell line. Nivolumab is a human IgG4 monoclonal antibody that binds to the PD-1 receptor and blocks its interaction with PD-L1 and PD-L2, releasing PD-1 pathway-mediated inhibition of the immune response, including the anti-tumor immune response. In syngeneic mouse tumor models, blocking PD-1 activity resulted in decreased tumor growth.

The preferred dose/amount of nivolumab is 240 mg. Preferably this amount is administered as an IV infusion. This amount is typically administered over 30 minutes every 2 weeks.

Nivolumab is indicated for unresectable or metastatic melanoma, adjuvant treatment of melanoma, neoadjuvant treatment of resectable NSCLC, metastatic NSCLC, malignant pleural mesothelioma, advanced renal cell carcinoma, classical Hodgkin lymphoma, recurrent or metastatic squamous cell carcinoma of the head and neck, urothelial carcinoma, microsatellite instability-high or mismatch repair deficient metastatic colorectal cancer, hepatocellular carcinoma, esophageal cancer, and gastric cancer, gastroesophageal junction cancer, and esophageal adenocarcinoma.

### Treatment Regimen

In one aspect, a IL-15 protein or fusion protein derivative thereof, or an IL-15:IL-15Rα protein or fusion protein derivative thereof, as well as a PD-1 inhibitor antibody and a taxane, are each administered systemically, for example, each are formulated in a pharmaceutically-acceptable buffers such as physiological saline. Preferable routes of administration include, for example, subcutaneous (SC), intravenous (IV), intraperitoneal, intramuscular, or intradermal injections and/or infusions.

Compositions comprising a IL-15 protein or fusion protein derivative thereof, or an IL-15:IL-15Rα protein or fusion protein derivative thereof, as well as compositions comprising a PD-1 inhibitor antibody and/or a taxane for parenteral use may be provided in unit dosage forms (e.g., each in single-dose ampoules, syringes or bags), or each in vials containing several doses and in which a suitable preservative may be added. The compositions may be in the form of a solution, a suspension, an emulsion, an infusion device, or a delivery device for implantation, or it may be presented as a dry powder to be reconstituted with water or another suitable vehicle before use. Furthermore, the compositions may include suspending, solubilizing, stabilizing, pH-adjusting agents, tonicity adjusting agents, and/or dispersing, agents.

In one aspect, the subject is initially administered a first cycle of treatment comprising administering an effective amount of a IL-15 protein or fusion protein derivative thereof, or an IL-15:IL-15Rα protein or fusion protein derivative thereof and/or composition comprising an effective amount of a IL-15 protein or fusion protein derivative thereof, or an IL-15:IL-15Rα protein or fusion protein derivative thereof subcutaneously to the subject, followed by administering an effective amount of a PD-1 inhibitor antibody and/or a composition comprising an effective amount of a PD-1 inhibitor antibody intravenously to the subject, followed by administering an effective amount of a taxane and/or a composition comprising an effective amount of a taxane intravenously to subject.

In a further aspect, the first cycle of treatment is followed by a second cycle of [0110] treatment comprising further administering an effective amount of a IL-15 protein or fusion protein derivative thereof, or an IL-15:IL-15Rα protein or fusion protein derivative thereof and/or a composition comprising an effective amount of a IL-15 protein or fusion protein derivative thereof, or an IL-15:IL-15Rα protein or fusion protein derivative thereof subcutaneously to the subject, followed by administering an effective amount of a PD-1 inhibitor antibody and/or a composition comprising an effective amount of a PD-1 inhibitor antibody intravenously to the subject, followed by administering an effective amount of a taxane and/or a composition comprising an effective amount of a taxane intravenously to subject, 19 to 23 days after completion of the first treatment cycle.

In yet another aspect, the subject is administered a third cycle of treatment comprising further administering an effective amount of a IL-15 protein or fusion protein derivative thereof, or an IL-15:IL-15Rα protein or fusion protein derivative thereof and/or a composition comprising an effective amount of a IL-15 protein or fusion protein derivative thereof, or an IL-15:IL-15Rα protein or fusion protein derivative thereof subcutaneously to the subject, followed by administering an effective amount of a PD-1 inhibitor antibody and/or a composition comprising an effective amount of a PD-1 inhibitor antibody intravenously to the subject, 40 to 44 days after the completion of the first cycle.

In each of the treatment cycles, the effective amount of the IL-15 protein or fusion protein derivative thereof, or the IL-15:IL-15Rα protein or fusion protein derivative thereof is 1.2 mg. In a preferred aspect, the administered IL-15:IL-15Rα fusion protein derivative is an IL-15N72D:IL-15RαSu/Fc complex (N-803). In one aspect, the subject is administered pharmaceutical composition comprising the IL-15 protein or fusion protein derivative thereof, or an IL-15:IL-15Rα protein or fusion protein derivative thereof. In one aspect, the subject is administered a pharmaceutical composition comprising N-803 formulated for administration by subcutaneous injection.

In each of the treatment cycles, the effective amount of the PD-1 inhibitor antibody is 200 mg. In one aspect, the PD-1 inhibitor antibody is tislelizumab, nivolumab, pembrolizumab, MEDI0680 or cemiplimab. In a preferred aspect, the PD-1 inhibitor antibody is tislelizumab. In one aspect, the subject is administered a pharmaceutical composition comprising the PD-1 inhibitor antibody. In one aspect, the subject is administered a pharmaceutical composition comprising tislelizumab.

In each of the treatment cycles the effective amount of the taxane is in an amount of 75 mg/m². In a preferred aspect, the taxane is docetaxel. In one aspect, the subject is administered a pharmaceutical composition comprising the taxane. In one aspect, the subject is administered a pharmaceutical composition comprising docetaxel.

In each treatment cycle, each of the IL-15 or variant thereof, the PD-1 inhibitor antibody and the taxane are formulated into separate pharmaceutical compositions.

### Kits

Pharmaceutical compositions comprising IL-15 protein or fusion protein derivative thereof, or an IL-15:IL-15Rα protein or fusion protein derivative thereof, and a PD-1 inhibitor antibody and a taxane, may be assembled into kits or pharmaceutical systems for use in treating a NSCLC. Kits or pharmaceutical systems according to this aspect of the invention comprise a carrier means, such as a box, carton, tube, having in close confinement therein one or more container means, such as vials, tubes, ampoules, bottles, syringes, or bags. In one aspect, the kit comprises a first vile comprising IL-15 protein or fusion protein derivative thereof, or an IL-15:IL-15Rα protein or fusion protein derivative thereof, wherein the composition is formulated for subcutaneous administration to the subject; a second vile comprising a PD-1 inhibitor antibody formulated for intravenous administration to the subject; a third vile comprising a taxane formulated for intravenous administration to the subject; and instructions for the use thereof.

In one aspect of the kit, the IL-15:IL-15Rα fusion protein derivative is an IL-15N72D:IL-15RαSu/Fc complex (N-803), wherein the N-803 comprises a dimeric IL-15RαSu/Fc and two IL-15N72D molecules. In one aspect of the kit, the N-803 is in an amount of 1.2 mg.

In one aspect of the kit, the PD-1 inhibitor antibody is tislelizumab. In one aspect of the kit, the PD-1 inhibitor antibody is tislelizumab and is in an amount of 200 mg.

In one aspect of the kit, the taxane is docetaxel. In one aspect of the kit, the taxane is docetaxel and is in an amount of 20 mg per 2 ml single dose vial, 80 mg per 8 mL multiple dose vial or 160 mg per 16 mL multiple dose vial. In one aspect, the docetaxel is formulated for administration at an amount of 75 mg/m².

The kits or pharmaceutical systems of the invention may also comprise associated instructions for using IL-15 protein or fusion protein derivative thereof, or an IL-15:IL-15Rα protein or fusion protein derivative thereof including N-803.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. It will be appreciated that, although not precluded, treating a disorder or condition does not require that the disorder, condition or symptoms associated therewith be completely eliminated.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

### The Mechanism of Action of N-803 can be Summarized as Follows:

The N-803 fusion protein includes 2 IL-15 receptor α-Sushi domains that bind and retain IL-15 with an N72D mutation (IL-15N72D). N-803 provides a soluble form of the IL-15-IL-15 receptor α-chain. It enables IL-15 signaling through the IL-2/IL-15 common receptor β γc complex without the need for monocyte or dendritic cell transpresentation; IL-15N72D is trans-presented by the IL-15 receptor α-chain to the shared IL-2/IL-15 receptor β and common γ (γc) chains displayed on the surface of CD4+ and CD8+ T cells and NK cells (Zhu 2009). The IL-15 receptor α-chain is expressed on monocytes and dendritic cells. In contrast, IL-2 receptor α-chain is expressed on the same cells that express IL-2/IL-15 receptor β and γc chains and thus IL-2 is presented in cis, not trans, to the receptor.

The inclusion of an IgG Fc domain in the N-803 fusion protein extends the half-life of N-803, an effect that may be due to interactions with the FcRn.

N-803 activates CD4+ and CD8+ T cells and NK cells, but not immunosuppressive regulatory T (Treg) cells (Yang 2020).

N-803 induces CD8+CD44high memory T cells to expand natural killer cells (Kim 2016).

### IL-15N72D Mutant Protein Increases Affinity for the IL-2/IL-15 Common Receptor.

The combination of N-803 and SoC ICIs in NSCLC offers a unique opportunity to overcome ICI resistance mediated by more than one mechanism, specifically, T cell exhaustion and HLA modulation. The hallmark of the CD8+ T-cell response in cancer or chronic viral infection is exhaustion, a dysfunctional state occurring as an adaptation to chronic antigen exposure (Bucks 2009). During exhaustion, persisting CD8+ T cells lose effector functions leading to a state of reduced efficacy (Ahmadzadeh 2009, Wherry 2003, Zajac 1998). It has been shown that the proliferative burst after PD-1 blockage came almost exclusively from infected-tissue-infiltrated exhausting PD-1+CD8+ cells in a chronic lymphocytic choriomeningitis virus infection model in mice (Im 2016). The effector CD8+ cells provide robust antiviral activities after the PD-1 blockage but become exhausted again shortly after PD-1 blockage, likely due to the lack of immune cell stimulation. This provides a strong scientific rationale to evaluate ANTKIVA plus an anti-PD-1 or anti-PD-L1 checkpoint inhibitor as combination therapy for individuals with NSCLC who have relapsed after achieving an initial response to PD-1 or PD-L1 blockade therapy.

In individuals with cancer, HLAs present tumor antigens, which are recognized by T cells which identify these cells as tumor. Classical HLA class I molecules are recognized by tumor-specific cytotoxic CD8+T lymphocytes, thus the downregulation or loss of HLA class I molecules enables tumors to escape from T-cell-mediated immune responses (Lo Monaco E., et al. Human leukocyte antigen E contributes to protect tumor cells from lysis by natural killer cells. Neoplasia. 2011; 13:822-30). Reduction in major histocompatibility complex (MHC) expression on acute myeloid leukemia (AML) cells is also a mechanism of immune evasion. RNA sequencing analysis of paired AML samples collected at diagnosis and at relapse post-transplantation identified altered expression of immune-related genes, including decreased expression of class II transactivator (CIITA), the master regulator of MHC-II expression, and of MHC-II molecules at relapse (Christopher M J., et al. Immune Escape of Relapsed AML Cells after Allogeneic Transplantation. N Engl J Med. 2018; 379:2330-41).

While classic HLA loss will prevent T-cell recognition in individuals with cancer, the expression of HLA-E molecules is enhanced. HLA-E functions as an immunomodulatory molecule by binding to the receptors CD94/NKG2A, -B, and -C on NK and T cells and functions effectively as a new immune checkpoint inhibitor (Braud V M., et al. HLA-E binds to natural killer cell receptors CD94/NKG2A, B and C. Nature. 1998; 391:795-9). N-803 may overcome this checkpoint resistance through activation and proliferation of NK cells up to 8-fold thereby potentially overwhelming the inhibitory checkpoint signals (Romee R., et al. 2018).

### N-803 Rescues T Cells and Checkpoint Inhibitors Through its Activation of Natural Killer Cells and IFN-γ Upregulation of MHC-I and MHC-II

Immune checkpoint inhibitors (ICIs) have revolutionized cancer therapy by reactivating T cells to target and eliminate tumor cells (Genova C., et al. Therapeutic Implications of Tumor Microenvironment in Lung Cancer: Focus on Immune Checkpoint Blockade. Front Immunol. 2021; 12:799455). However, the efficacy of ICIs relies heavily on the ability of tumor cells to present antigens through MHC-I and MHC-II (Jenkins R W., et al. Mechanisms of resistance to immune checkpoint inhibitors. Br J Cancer. 2018; 118:9-16; Passaro A., et al. Managing Resistance to Immune Checkpoint Inhibitors in Lung Cancer: Treatment and Novel Strategies. J Clin Oncol. 2022; 40:598-610), key molecules that enable T cells to recognize and attack malignant cells. Tumor evasion of the immune system, particularly through downregulation or loss of MHC-I, presents a significant barrier to effective therapy (Dhatchinamoorthy K., et al. Cancer Immune Evasion Through Loss of MHC Class I Antigen Presentation. Front Immunol. 2021; 12:636568). N-803 (N-803), an IL-15 superagonist, has emerged as a pivotal agent in overcoming these challenges by stimulating NK cells and enhancing T cell function through the upregulation of MHC-I and MHC-II via IFN-γ secretion (Ellis-Connell A L., et al. et al. IL-15 Superagonist N-803 Enhances IFNgamma Production of MAIT Cells in SIV (+) Macaques. Infect Immun. 2022; 90:e0025922; Knudson K M., et al. Mechanisms involved in IL-15 superagonist enhancement of anti-PD-L1 therapy. J Immunother Cancer. 2019; 7:82; Wong H C, et al. The IL-15-based superagonist ALT-803 promotes the antigen-independent conversion of memory CD8 (+) T cells into innate-like effector cells with antitumor activity. Oncoimmunology. 2013; 2:e26442).

### The Role of MHC-I and MHC-II in T Cell Activation and Tumor Immunity

MHC-I molecules are expressed on nearly all nucleated cells and are crucial for presenting intracellular antigens, such as those derived from viruses or tumor cells, to CD8+ killer T cells. When CD8+ T cells recognize antigens presented by MHC-I, they become activated and initiate a cytotoxic response, killing the malignant or infected cells (Wen M., et al. Insight into Cancer Immunity: MHCs, Immune Cells and Commensal Microbiota. Cells. 2023; 12). This MHC-I-driven recognition is a critical element of immune surveillance and is essential for the destruction of tumor cells. Without MHC-I expression, CD8+ T cells are unable to identify tumor cells, leading to immune escape (Dhatchinamoorthy K. et al. 2021).

MHC-II molecules, on the other hand, are primarily found on professional antigen-presenting cells like dendritic cells, macrophages, and B cells (Roche P A., et al. The ins and outs of MHC class II-mediated antigen processing and presentation. Nat Rev Immunol. 2015; 15:203-16). These molecules present extracellular antigens to CD4+ helper T cells, which are central to coordinating the broader adaptive immune response. Once activated by MHC-II, CD4+ T cells secrete cytokines, such as IFN-γ, that help amplify the immune response, supporting both the activation of CD8+ T cells and NK cells, and promoting the maturation of B cells. Thus, MHC-II is vital for sustaining a robust and coordinated immune response to tumors.

### MHC-I Loss and Tumor Evasion During ICI Therapy

One of the most common mechanisms of tumor immune evasion is the loss or downregulation of MHC-I on tumor cells. Without MHC-I expression, CD8+ T cells cannot recognize or kill the tumor cells, leading to unchecked tumor growth and progression (Sari G., et al. Tumor immune evasion through loss of MHC class-I antigen presentation. Curr Opin Immunol. 2023; 83:102329). This MHC-I loss results in the development of MHC-negative tumors, which are often resistant to ICI therapy and correspond to cold tumor microenvironments that lack immune cell infiltration. These cold tumors present a significant challenge to immunotherapies, as the fundamental machinery of T cell recognition is compromised.

In this context, MHC-I loss undermines the core mechanism of ICIs, which rely on reinvigorating T cell activity. Without MHC-I to present antigens to CD8+ T cells, the immune system's cytotoxic response is severely diminished, allowing the tumor to evade immune detection and therapy.

### The Role of IFN-γ in Upregulating MHC-I and MHC-II

IFN-γ plays a central role in enhancing the immune system's ability to target and eliminate tumor cells. One of its most critical functions is the upregulation of both MHC-I and MHC-II on tumor cells and antigen-presenting cells (APCs) (Zhou X., et al. Mechanisms of tumor resistance to immune checkpoint blockade and combination strategies to overcome resistance. Front Immunol. 2022; 13:915094). Through the action of IFN-γ, MHC-I expression is restored on tumor cells, allowing CD8+ T cells to once again recognize and destroy malignant cells. Simultaneously, IFN-γ upregulates MHC-II expression on APCs, ensuring that CD4+ T cells are activated to coordinate and amplify the immune response.

In cold tumors where MHC-I expression has been lost, the reintroduction of MHC-I through IFN-γ signaling is vital for reversing immune evasion and re-establishing the effectiveness of checkpoint inhibitors. Similarly, the upregulation of MHC-II ensures that APCs can activate CD4+ helper T cells, driving a robust immune response that includes the activation of both CD8+ killer T cells and NK cells.

### N-803's Mechanism: Activating NK Cells and Promoting IFN-γ Secretion

N-803 (N-803) is an IL-15 superagonist that targets the IL-15 receptor on NK cells and CD8+ T cells, leading to their proliferation and activation. IL-15 plays a critical role in enhancing the survival and cytotoxic activity of NK cells, which are essential in targeting MHC-negative tumors that escape CD8+ T cell recognition. N-803 stimulates NK cells to target and kill these tumors through a mechanism independent of MHC-I (Wong H C., et al. 2013).

Additionally, NK cells activated by N-803 produce high levels of IFN-γ, which is crucial for the upregulation of MHC-I and MHC-II on tumor cells and APCs, respectively. This IFN-γ-driven upregulation transforms cold tumors (lacking MHC expression) into hot tumors, where the reactivated CD8+ T cells can now recognize and destroy the malignant cells. In this way, N-803 restores immune surveillance and rescues the efficacy of checkpoint inhibitors by enabling CD8+ T cells to function effectively against previously resistant tumors (Lui G., et al. 2023).

### MHC-negative Tumors and the Universal Targeting by N-803

While MHC-I loss shields tumors from T cell-mediated destruction, it exposes them to NK cell mediated cytotoxicity. NK cells are programmed to eliminate cells that lack MHC-I, making MHC-negative tumors a universal target for N-803-activated NK cells. This bypasses the need for MHC-I-dependent antigen presentation and allows for the immune system to effectively target a broad range of MHC-negative tumor types. By activating NK cells and promoting IFN-γ secretion, N-803 ensures that tumors, even those resistant to ICIs, can be attacked and destroyed.

The unique ability of N-803 to proliferate NK cells and drive IFN-γ secretion addresses one of the primary mechanisms of tumor resistance—MHC-I loss—by upregulating MHC-I and MHC-II, converting immune-resistant cold tumors into immune-responsive hot tumors. This reactivation of both innate and adaptive immune pathways ensures a comprehensive anti-tumor response.

### Restoring Immune Function and Prolonging Survival

N-803's dual action—activating NK cells and enhancing T cell function through IFN-γ-mediated upregulation of MHC-I and MHC-II—restores immune function in patients with MHC-negative tumors. By bridging the innate and adaptive arms of the immune system, N-803 ensures that tumors are targeted from multiple angles, promoting long-term immune memory and durable remission. The proliferation of NK cells and CD8+ T cells, combined with the reactivation of MHC-I and MHC-II, enables checkpoint inhibitors to regain their full potency, driving more effective and prolonged responses in cancer therapy.

### N-803 Rescues T Cells and Checkpoint Inhibitors Through its Activation of Natural Killer Cells and IFN-γ Upregulation of MHC-I and MHC-II

N-803 leverages the power of IL-15 to activate NK cells and rescue CD8+ T cell function through IFN-γ-mediated upregulation of MHC-I and MHC-II. By converting MHC-negative (cold) tumors into MHC+ (hot) tumors, N-803 restores immune surveillance and enhances the efficacy of checkpoint inhibitors (AACR 2018, Wrangle 2020). Through its ability to upregulate key MHC molecules, N-803 ensures that both NK cells and T cells can effectively target and destroy tumors, leading to prolonged survival and durable remission in patients with cancer. This multi-pronged approach addresses a critical mechanism of tumor resistance, offering a promising pathway for overcoming ICI resistance and driving robust immune responses.

### N-803 and Immune Checkpoint Inhibitor Combination Therapy:

Disclosed herein is a treatment regimen designed to maximize ICD and augment and maintain the innate and adaptive immune responses against cancer cells. The IL-15 superagonist, N-803, is used in combination with an ICI to enhance tumor-specific cytotoxic T-cell responses and the activity of endogenous NK cells in individuals with NSCLC who have relapsed after achieving an initial response to PD-1 or PD-L1 blockade therapy.

Given the immune activating properties of N-803 and the potential to complement ICI therapy, animal studies were conducted to examine this combination therapy. Mice bearing established 5T33P and colon tumor 26 (CT26) tumors treated with N-803 in combination with anti-PD-L1 and anti-cytotoxic T-lymphocyte-associated protein 4 (CTLA4), were found to have prolonged survival when compared to monotherapy (Kim 2016). In a glioblastoma 261-luciferase mouse model (GL261-luc) administration of N-803 (either 3 doses or 4 doses) or anti-PD-1 antibody (3 doses) produced 20% of long-term survivor mice (>60 days post-implantation), and their combination increased to 40% long-term survival (Mathios 2016). Based on these results, preclinical data human studies were conducted.

In ongoing studies, combination therapy with N-803 has been well-tolerated. An interim analysis of QUILT-3.055 (NCT03228667) examined clinical response and safety in patients with 2nd and 3rd line+ NSCLC (as part of a basket trial of multiple tumor types) who were previously treated and failed either ICI alone or failed ICI in combination with chemotherapy. These patients had received no intervening therapy. Patients received N-803 15 μg/kg subcutaneously every 3 weeks in combination with the same checkpoint inhibitor they previously received and on which they had progressed.

Phase 2 data presented at the World Conference on Lung Cancer in 2024 showed the median OS (n=86 NSCLC patients) was 14.1 months (95% CI 11.7, 17.4) with survival ranging up to 58 months. Overall survival (OS) for PD-L1+ve (>1%) (N=53) was 13.8 months (95% CI 10.2, 16.2) versus PD-L1−ve (N=33) of 15.8 months (95% CI 11.5, 24.0). The N-803 adverse event profile was consistent with ICI alone with no cytokine release syndrome observed. Only 10% of participants had any grade ≥3 N-803 related AEs. The study demonstrated long-term survival at ≥12 and ≥18 months of 49/86 (57%) and 29/86 (34%) patients respectively. These data continue to validate the mechanism of action of N-803 in activating NK, CD8 killer, and Memory T cells resulting in prolonged overall survival in patients with advanced cancers and support the ResQ201A-NSCLC trial concept.

The following examples are put forth so as to provide those of ordinary skill in the art with a complete disclosure and description of how to make and use the assay, screening, and therapeutic methods of the invention, and are not intended to limit the scope of what the inventors regard as their invention. All publications, patents, and patent applications cited herein are hereby incorporated by reference in their entirety for all purposes.

### EXAMPLE

This example discloses a randomized, open-label, phase 3 clinical trial to compare the efficacy and safety of N-803 plus tislelizumab and docetaxel (experimental arm) versus docetaxel monotherapy (control arm).

Study Design: Enrolled subjects are randomized 2:1 to treatment in the experimental arm or the control arm. Subject randomization is stratified by geographical region (North America vs Europe vs ASIA), NSCLC histology (squamous vs nonsquamous), and actionable genomic alteration (AGA); (epidermal growth factor receptor [EGFR]/anaplastic lymphoma kinase [ALK] reactive oxygen species [ROS] vs OTHER AGA vs No AGA).

Screening (within 28 days prior to baseline) and baseline: The screening period is the 28-day period prior to baseline. During the screening period, subjects' eligibility for the study is determined. Baseline is defined as study day 1 prior to start of first study treatment.

### Treatment Period

Experimental Arm: The experimental arm treatment regimen consists of an induction phase of 2, 3-week cycles followed by a maintenance phase of at least 3, 3-week cycles with a ±3-day window for each visit. Agents are administered in the order listed (e.g., on day 1 experimental arm [cycles 1 and 2], administer N-803, followed by tislelizumab and then followed by docetaxel). The treatment regimen for the experimental arm is as follows:
Induction Phases: Cycles 1 and 2 (3-week cycles):
   Cycle 1 and 2 Day 1:
      N-803 1.2 mg SC
      Tislelizumab 200 mg IV
      Docetaxel 75 mg/m² IV
   Maintenance Phase: Cycles ≥3 (repeated 3-weeks cycles):
   Day 1:
      N-803 1.2 mg SC
      Tislelizumab 200 mg IV

Control Arm: The control arm treatment regimen consists of repeated 3-week cycles with a ±3-day window for each visit. The treatment regimen for all cycles in the control arm is as follows.
Day 1:
   Docetaxel 75 mg/m² IV

An example of the treatment schedules and order of administration are provided in Tables 1, 2 and 3.

TABLE 1
Schedule and Order of Administration for the Experimental Arm (Cycles 1 to 2)
Experimental Arm Induction Treatment Period (3-week cycles)^{a}

| | Cycle | | | | | | | | | | | | | | | | | | | | |
|---|---|---|---|---|---|---|---|---|---|---|---|---|---|---|---|---|---|---|---|---|---|
| | Cycle 1 | | | | | | | | | | | | | | | | | | | | |
| | Cycle Week | | | | | | | | | | | | | | | | | | | | |
| | 1 | | | | | | | 2 | | | | | | | 3 | | | | | | |
| | Cycle Day | | | | | | | | | | | | | | | | | | | | |
| | 1 | 2 | 3 | 4 | 5 | 6 | 7 | 8 | 9 | 10 | 11 | 12 | 13 | 14 | 15 | 16 | 17 | 18 | 19 | 20 | 21 |
| | Study Visit Window (days) | | | | | | | | | | | | | | | | | | | | |
| | ±3 | | | | | | | | | | | | | | | | | | | | |
| N-803 | X | | | | | | | | | | | | | | | | | | | | |
| 1.2 mg SC | | | | | | | | | | | | | | | | | | | | | |
| Tislelizumab | X | | | | | | | | | | | | | | | | | | | | |
| 200 mg IV | | | | | | | | | | | | | | | | | | | | | |
| Docetaxel | X | | | | | | | | | | | | | | | | | | | | |
| 75 mg/m² IV | | | | | | | | | | | | | | | | | | | | | |
| | Cycle | | | | | | | | | | | | | | | | | | | | |
| | Cycle 2 | | | | | | | | | | | | | | | | | | | | |
| | Cycle Week | | | | | | | | | | | | | | | | | | | | |
| | 1 | | | | | | | 2 | | | | | | | 3 | | | | | | |
| | Cycle Day | | | | | | | | | | | | | | | | | | | | |
| | 1 | 2 | 3 | 4 | 5 | 6 | 7 | 8 | 9 | 10 | 11 | 12 | 13 | 14 | 15 | 16 | 17 | 18 | 19 | 20 | 21 |

| | Study Visit Window (days) |
|---|---|
| | ±3 |
| N-803 | X |
| 1.2 mg SC | |
| Tislelizumab | X |
| 200 mg IV | |
| Docetaxel | X |
| 75 mg/m² IV | |

^{a}Agents are to be administered in the order listed in the table (e.g., on day 1 experimental arm [cycle 1], administer N-803, followed by tislelizumab and then followed by docetaxel).

TABLE 2
Treatment Schedule and Order of Administration (Experimental Arm Cycles ≥3)
Experimental Arm Maintenance Treatment Period (repeated 3-week cycles)^{a,b}

| Cycle | | | | | | | | | | | | | | | | | | | | |
|---|---|---|---|---|---|---|---|---|---|---|---|---|---|---|---|---|---|---|---|---|
| Cycles ≥3 | | | | | | | | | | | | | | | | | | | | |
| Cycle Week | | | | | | | | | | | | | | | | | | | | |
| 1 | | | | | | | 2 | | | | | | | 3 | | | | | | |
| Cycle Day | | | | | | | | | | | | | | | | | | | | |
| 1 | 2 | 3 | 4 | 5 | 6 | 7 | 8 | 9 | 10 | 11 | 12 | 13 | 14 | 15 | 16 | 17 | 18 | 19 | 20 | 21 |

| | Study Visit Window (days) |
|---|---|
| | ±3 |
| N-803 | X |
| 1.2 mg SC | |
| Tislelizumab | X |
| 200 mg IV | |

^{a}Agents are administered in the order listed in the table (e.g., on day 1 experimental arm [cycles ≥3], administer N-803 followed by tislelizumab
^{b}Subjects receive treatment in the experimental arm until iCPD, unacceptable toxicity, withdrawal of consent, or if the Investigator feels it is no longer in their best interest to continue treatment.

TABLE 3
Treatment Schedule and Order of Administration for the Control Arm

| Treatment Period (Repeated, 3-week cycles)^{a} | | | | | | | | | | | | | | | | | | | | |
|---|---|---|---|---|---|---|---|---|---|---|---|---|---|---|---|---|---|---|---|---|
| Cycle | | | | | | | | | | | | | | | | | | | | |
| Cycle 1 | | | | | | | | | | | | | | | | | | | | |
| Cycle Week | | | | | | | | | | | | | | | | | | | | |
| 1 | | | | | | | 2 | | | | | | | 3 | | | | | | |
| Cycle Day | | | | | | | | | | | | | | | | | | | | |
| 1 | 2 | 3 | 4 | 5 | 6 | 7 | 8 | 9 | 10 | 11 | 12 | 13 | 14 | 15 | 16 | 17 | 18 | 19 | 20 | 21 |

| | Study Visit Window (days) |
|---|---|
| | ±3 |
| Docetaxel | X |
| 75 mg/m² IV | |

^{a}Subjects receive treatment in the control arm until iCPD, unacceptable toxicity, withdrawal of consent, or if the Investigator feels it is no longer in their best interest to continue treatment

N-803 (also known as ALT-803) is in a liquid formulation that appears clear to opalescent, colorless to slightly yellow. It is in a 2-mL vial containing 0.6 mL of N-803 at a concentration of 2 mg/ml. It is stored between 2-8° C. The preferred injection site for N-803 SC administration is in the abdominal area. The preferred specific injection location is the lower left or right quadrant of the abdomen, alternating sides between doses. After injection, the participant is closely monitored for N-803 injection reactions.

Tislelizumab (200 mg) is in a liquid formulation that appears clear to slightly opalescent, colorless to slightly yellow and is administered as a 30-minute IV infusion (an initial 60-minute IV infusion may occur followed by subsequent 30-minute IV infusions). It is preferably in a single-use vial containing a total of 100 mg of antibody in 10 mL of isotonic solutions. After infusion the participant is closely monitored for infusion-related reactions.

Doxcetaxel (75 mg/m2) is administered by IV infusion over 1-hour. After infusion the participant is closely monitored for infusion-related reactions.

Dose modifications for toxicity resulting from the nonapproved investigational products in the treatment regimen is initiated after completion of the first cycle of treatment and are relative to the dose administered when the toxicity occurred. Dose modifications for toxicities resulting from the approved investigational products in the treatment regimen are allowed according to the applicable Health authority approved product label.

In the event dosing is delayed >2 weeks, the delayed dose should be skipped and resumed at the next scheduled treatment.

### Experimental and Control Arms

Subjects receive treatment until confirmed disease progression per iRECIST (immune confirmed progressive disease [iCPD]), unacceptable toxicity, withdrawal of consent, or it is no longer in their best interest to continue treatment.

Tumors are assessed at screening, and tumor response is assessed at week 6, week 12, week 18, and every 12 weeks (+1 week) thereafter by computed tomography (CT) or magnetic resonance imaging (MRI) of target and nontarget lesions in accordance with RECIST v1.1 and iRECIST. The imaging schedule is established at the initiation of randomization and followed during the study regardless of any dosing delays or modifications to the timing of other assessments. In order to document progressive disease (PD), unscheduled tumor assessments may be done if the Investigator observes any signs and symptoms of PD. If disease progression per RECIST v1.1 is initially observed, an imaging assessment is done 4-8 weeks after the initial PD assessment to rule out tumor pseudoprogression. If confirmation of PD per iRECIST (iCPD) is not observed, participants are allowed to continue treatment and response assessments will continue at week 6, week 12, week 18, and every 12 weeks thereafter and will continue to be evaluated per iRECIST. Treatment is discontinued upon iCPD however, treatment may continue beyond iCPD per iRECIST as long as the subject is clinically stable and is considered to be deriving clinical benefit.

Safety is assessed for all subjects and includes clinically significant changes in laboratory tests and vital signs and the frequency and severity of AEs graded using National Cancer Institute (NCI) Common Terminology Criteria for Adverse Events (CTCAE) v5.0.

### End-of-Treatment Visit (30 [+7] Days Post Last Dose):

The end-of-treatment (EOT) visit occurs 30 (+7) days following the subject's last dose of treatment.

### Posttreatment Follow-Up Period (After End-of-Treatment Visit):

The follow-up period begins after the EOT visit. Subjects who discontinue study treatment remain in the study for follow-up. All subjects are followed for collection of posttreatment therapies and survival status every 3 months (+6 weeks) until either death (any cause) or for a maximum of 36 months post administration of the last dose of study drug. Imaging during follow-up occurs as per standard of care.

### Number of Subjects:

The study is planned for up to 578 subjects to be screened in order for 462 subjects to be randomized 2:1 to the experimental arm or control arm (i.e., 308 participants to the experimental arm and 154 participants to the control arm).

### Duration of Study:

Subjects receive treatment until iCPD, unacceptable toxicity, withdrawal of consent, or if it is no longer in their best interest to continue treatment. Treatment may be discontinued upon confirmed PD per iRECIST; however, treatment may continue beyond confirmed PD per iRECIST if the participant is clinically stable and is considered to be deriving clinical benefit.

### Duration of Follow-Up:

The follow-up period begins after the EOT visit. Subjects who discontinue study treatment remain in the study for follow-up. Subjects who discontinue treatment for reasons other than PD are followed for CT or MRI imaging and response assessment at week 6, week 12, week 18, and every 12 weeks thereafter (+1 week) until disease progression is confirmed per iRECIST. All subjects are followed (telemedicine acceptable) for collection of poststudy therapies and survival status every 3 months (+6 weeks) until either death (any cause) or for a maximum of 36 months post administration of the last dose of study drug.

### Study Endpoints:

Response assessments for the primary and secondary endpoints are based on local review if not specified otherwise.

### Primary Endpoint:

OS, as defined as time from randomization to death resulting from any cause.

### Secondary Endpoints:

### Efficacy:

iDCR, (confirmed immune complete response [iCR], immune partial response [iPR], or immune stable disease [iSD] lasting for at least 2 months) as measured using iRECIST.

iPFS, defined as time from randomization to disease progression or death from any cause, whichever occurs first as measured using iRECIST.

iORR and iDOR as measured using iRECIST.

### Safety:

AEs and SAEs graded using the NCI CTCAE v5.0.

Clinically important changes in laboratory tests and vital signs.

### Exploratory Endpoints:

### Efficacy:

DSS, defined as, time from randomization to death resulting from NSCLC.

DCR, PFS, ORR, DOR as measured using RECIST v1.1.

### Other:

Whole slide images and correlations with participant outcomes.

Blood for analyses which may include assessment of ctDNA.

Tumor molecular profiles and their correlations with participant outcomes.

### Participant Enrollment and Withdrawal:

Participate Eligibility: Women and men of all races and ethnic groups are eligible for this trial.

### Inclusion Criteria

Participants must meet ALL of the following criteria for inclusion in the study:
1. Age ≥18 years old.
2. Able to understand and provide a signed informed consent that fulfills the relevant Institutional Review Board (IRB) or Independent Ethics Committee (IEC) guidelines.
3. Pathologically confirmed stage IV NSCLC disease.
4. Have acquired resistance to an immune checkpoint inhibitor, defined as disease progression immediately following an initial response or stable disease (≥6 months duration) to exactly 1 line of anti-PD-L1 or anti-CTLA-4 therapy (for Stage III, IV, or recurrent disease) that was given alone or in combination with chemotherapy.
5. Participants with AGA must have 1 or more documented AGA(s): EGFR, ROS1, neurotrophic tyrosine receptor kinase (NTRK), B rapidly accelerated fibrosarcoma (BRAF), mesenchymal epithelial transition (MET) exon 14 skipping, rearranged during transfection (RET), Kirsten Rat sarcoma (KRAS).
6. Participants with AGA must meet the following criteria for advanced or metastatic NSCLC.
7. Participants who have been treated with 1 or 2 prior lines of applicable targeted therapy that is locally approved (and is standard of care) for the participant's genomic alteration at the time of screening:
   a. Participants who have tumors with EGFR L858R or exon 19 deletion mutations must have received prior Osimertinib.
   b. Participants who received a targeted agent as adjuvant therapy for early-stage disease must have relapsed or progressed while on the treatment or within 6 months of the last dose or received at least one additional course of targeted therapy for the same genomic alteration (which may or may not be same agent used in the adjuvant setting) for relapsed/progressive disease.
   c. Participants who have been treated with a prior tyrosine kinase inhibitor (TKI) must receive additional approved targeted therapy, if locally available and clinically appropriate, for the applicable genomic alteration, or the participant will not be allowed in the study.
   d. Participants must meet the inclusion criteria #4 listed above. 7. ECOG performance status of 0 to 2.
8. Measurable tumor lesions according to RECIST v1.1.
9. Ability to attend required study visits and return for adequate follow-up, as required by this protocol.
10. Agreement to practice effective contraception for female participants of child-bearing potential and non-sterile males. Female participants of child-bearing potential must agree to use effective contraception for up to 7 months after completion of therapy, and non-sterile male participants must agree to use a condom for up to 7 months after treatment. Effective contraception includes surgical sterilization (e.g., vasectomy, tubal ligation), orals, injectables, 2 forms of barrier methods (e.g., condom, diaphragm) used with spermicide, intrauterine devices (IUDs), and hormonal therapy.
11. Participants with known HIV infection must be receiving anti-retroviral therapy and have an undetectable viral load at their most recent viral load test within 6 months prior to enrollment.

### Exclusion Criteria:

1. Systemic autoimmune disease currently requiring treatment (e.g., lupus erythematosus, rheumatoid arthritis, Addison's disease, or autoimmune disease associated with lymphoma). The participant must have been off treatment for 180 days.
2. History of organ transplant requiring immunosuppression; or history of pneumonitis or interstitial lung disease requiring treatment with systemic steroids; or a history of receiving systemic steroid therapy or any other immunosuppressive medication ≤3 days prior to study initiation. Daily steroid replacement therapy (e.g., prednisone or hydrocortisone) and corticosteroids used to manage AEs are permitted.
3. Participants with actionable genomic alteration (AGA) of ALK.
4. History of known active hepatitis B or C infection.
5. Active infection requiring antibiotic therapy.
6. History of or active inflammatory bowel disease (e.g., Crohn's disease, ulcerative colitis).
7. Participants with known history of severe hypersensitive reactions to docetaxel or to other drugs formulated with polysorbate 80.
8. Had major surgery within 28 days prior to study randomization. Participants must have fully recovered from the effects of prior surgery in the opinion of the treating Investigator.
9. Inadequate organ function, evidenced by the following laboratory results:
   a. Absolute lymphocyte count <institutional ULN.
   b. Absolute neutrophil count <1,500 cells/mm3.
   c. Platelet count <100,000 cells/mm3.
   d. Total bilirubin greater than the upper limit of normal (ULN; unless the participant has documented Gilbert's syndrome).
   e. Aspartate aminotransferase (AST [SGOT]) or alanine aminotransferase (ALT [SGPT])>1.5×ULN.
   f. Alkaline phosphatase (ALP) levels >2.5×ULN.
   g. Hemoglobin <9.0 g/dL.
   h. Serum creatinine >2.0 mg/dL or 177 μmol/L or creatinine clearance <40 mL/min (using the Cockcroft-Gault formula below):

$$Female = [ ( 140 - age in years ) \times weight in kg \times 0.85 ] / [ 72 \times serum creatinine in mg / dL ]$$

$$Male = [ ( 140 - age in years ) \times weight in kg \times 1. ] / [ 72 \times serum creatinine in mg / dL ]$$

10. Have any of following:
   a. Cirrhosis at a level of Child-Pugh B (or worse);
   b. Cirrhosis (any degree) and a history of hepatic encephalopathy; or
   c. Clinically meaningful ascites resulting from cirrhosis. Clinically meaningful ascites is defined as ascites from cirrhosis requiring diuretics or paracentesis.
11. Participation in an investigational drug study or history of receiving any investigational treatment within 30 days prior to the start of treatment on this study, except for hormone-lowering therapy in participants with hormone-sensitive cancers.
12. Assessed by the Investigator to be unable or unwilling to comply with the requirements of the protocol.
13. Pregnant and nursing women.
14. History of allergic reactions to tislelizumab.

### Tumor Response:

Participants (also referred to herein as subjects) are evaluated for tumor burden by CT or MRI imaging at screening (up to 28 days before enrollment). Subsequent evaluations for tumor response occur at week 6, week 12, week 18, then every 12 weeks thereafter (+1 week) following the first drug administration until the last drug administration based on calendar dates. Imaging occurs until iCPD by iRECIST. The imaging schedule is established at the initiation of treatment and followed during the treatment period regardless of any dosing delays or modifications to the timing of other assessments. Imaging during follow-up occurs as per standard of care.

Unscheduled confirmatory tumor assessments is done if any signs and symptoms of PD are observed. When disease progression per RECIST v1.1 is initially observed, an imaging assessment is done 4-8 weeks (+1 week) after the initial PD assessment to rule out pseudoprogression. If confirmation of PD per iRECIST is not observed, subjects continue treatment and response assessments continue on the original imaging schedule at week 6, week 12, week 18, then every 12 weeks (+1 week) thereafter and are evaluated per iRECIST. Evaluations may include CT or MRI scans of the chest, abdomen, pelvis (optional unless known pelvic disease is present at baseline), and brain (only as clinically warranted based on symptoms/findings).

Treatment is discontinued upon confirmed iCPD per iRECIST; however, treatment may continue beyond confirmed iCPD per iRECIST provided the following conditions are met: the subject is clinically stable and is considered to be deriving clinical benefit; absence of clinical symptoms and signs indicating clinically significant disease progression; no decline in participant PS; and absence of rapid disease progression or threat to vital organs or critical anatomical sites (e.g., central nervous system [CNS] metastasis, respiratory failure due to tumor compression, or spinal cord compression) requiring urgent alternative medical intervention.

Prior to enrollment, tumor lesions to be followed for response are clearly identified by location and selected and categorized as target or non-target lesions. Target lesions include those lesions that can be accurately measured in at least 1 dimension as ≥10 mm, using CT or MRI with a slice thickness ≤5 mm. Malignant lymph nodes with a short axis diameter ≥15 mm can be considered target lesions. Up to a maximum of 2 target lesions per organ and 5 target lesions in total will be identified at baseline. These lesions should be representative of all involved organs and selected based on their size (those with the longest diameter) and their suitability for accurate repeated measurements. A sum of the longest lesion diameter (LLD) for all target lesions are calculated and reported as the baseline sum LLD. For malignant lymph nodes identified as target lesions, the short axis diameter is used in the sum of LLD calculation. All other lesions (or sites of disease) are identified as non-target lesions (including bone lesions) (Eisenhauer E A., et al. New response evaluation criteria in solid tumors: revised RECIST guideline (version 1.1). Eur J Cancer. 2009; 45:228-47).

All postbaseline response assessments follow the same lesions identified at baseline. The same mode(s) of assessment (ie, CT or MRI) used to identify/evaluate lesions at baseline are used throughout the course of the study unless participant safety necessitates a change (e.g., allergic reaction to contrast media).

RECIST and iRECIST:

The primary assessment of antitumor activity is evaluation of target and/or non-target lesions in accordance with RECIST v1.1 (Eisenhauer E A., et al. 2009).

Since immunotherapy may cause an inflammatory response that could be interpreted as disease progression, the Investigator also classifies response in accordance with iRECIST (Seymour L et al. et al. iRECIST: guidelines for response criteria for use in trials testing immunotherapeutics. Lancet Oncol. 2017; 18:e143-e52).

### Response Assessments:

Percentage change in target lesion size will be evaluated by the following formulae:

When determining CR or PR:

$$( [ Post value - Baseline value ] / Baseline value ) \times 100$$

When determining PD:

$$( [ Post value - Smallest value since treatment started ] / [ Smallest value since treatment started ] ) \times 100$$

Response of target lesions is classified in accordance with RECIST v1.1. Response of nontarget lesions is classified in accordance with RECIST v1.1. Overall response is classified in accordance with RECIST v1.1.

Tumor response at each time point is classified per iRECIST. Best overall response is assigned per iRECIST.

### Circulating Tumor DNA Analyses:

Somatic alterations detected by ctDNA may be predictive of response to therapies, including immune checkpoint inhibitors (Anagnostou V., et al. ctDNA response after pembrolizumab in non-small cell lung cancer: phase 2 adaptive trial results. Nature Medicine. 2023; 29:2559-69; Zhang Q., et al. Prognostic and Predictive Impact of Circulating Tumor DNA in Patients with Advanced Cancers Treated with Immune Checkpoint Blockade. Cancer Discov. 2020; 10:1842-53). Dynamic changes in ctDNA quantity are also prognostic of response to therapies. Decrease or clearance of ctDNA quantity is associated with improved PFS. Identification of patients with increasing ctDNA may enable detection of patients who are likely to have molecular progression prior to clinical progression on CT scans. Blood will be collected (2×10 mL Streck tubes) from all participants at baseline, on day 1 of cycles 3 and 5 and at each subsequent CT scan (every 12 weeks, +1 week]) until EOT for plasma isolation and storage. Blood should be collected prior to any therapy given on the day of collection. ctDNA analysis will be performed to determine the landscape of somatic alterations in each sample and quantify dynamic changes in ctDNA, including ctDNA clearance.

### Statistical Considerations:

Descriptive statistics will be used to summarize the study endpoints. Unless otherwise stated, the term descriptive statistics refers to the number of participants, mean, median, standard deviation, minimum, and maximum for continuous data, and frequencies and percentages for categorical data. All confidence intervals (CIs) will be constructed at the two-sided 95% level of confidence.

Analysis of Primary Efficacy Endpoint: The primary efficacy endpoint is OS defined as time from randomization to death resulting from any cause and will be analyzed using Kaplan-Meier (KM) methods. Participants who are alive at the end of follow-up are censored in the OS analysis at the last known date alive. The KM curve and median OS (and 95% CI) are summarized for each treatment arm. Comparison of OS between the experimental arm and control arm is tested based on the stratified log-rank test, stratified by the randomization strata. The OS hazard ratio (HR) (and 95% CI) is summarized based on the stratified Cox proportional hazard model, stratified by the randomization strata.

Analysis of Secondary Efficacy Endpoints: The secondary efficacy endpoints (iDCR, iPFS, iORR, and iDOR) is tested only if the primary efficacy endpoint, OS, is statistically significant in favor of experimental arm. In order to control the type 1 error rate for multiple secondary endpoints, a hierarchical/gate-keeping approach is used to test the secondary endpoints. Each secondary endpoint is tested at α=0.05 according to the following hierarchy:
iDCR by iRECIS
iPFS by iRECIS
iORR by iRECIS
iDOR by iRECIST

Analysis of iDCR: iDCR is defined as a confirmed iCR, iPR, or iSD lasting for at least 2 months as measured using iRECIST. The iDCR (and 95% CI) is summarized for each treatment arm. Comparison of iDCR between the experimental arm and control arm, if tested, is based on the Cochran-Mantel-Haenszel (CMH) test, stratified by the randomization strata.

Analysis of iPFS: The iPFS is defined as the time from the date of the randomization to the date of iPD or death (any cause), whichever occurs first, and is analyzed using KM methods. Participants completing study follow-up or initiating a new anticancer therapy prior to iPD are censored at the last known date the participant was progression-free prior to completing follow-up or initiating the new therapy. The KM curve and median iPFS (and 95% CI) is summarized for each arm. Comparison of iPFS between the experimental arm and control arm, if tested, is based on the stratified log-rank test, stratified by the randomization strata. The iPFS HR (and 95% CI) is summarized based on the Cox proportional hazard model.

Analysis of iORR: The iORR is defined as participants with a confirmed response (iCR or iPR) with confirmation at least 4 weeks after the initial response. The iORR (and 95% CI) is summarized for each treatment arm. Comparison of iORR between the experimental arm and control arm, if tested, is based on the CMH test, stratified by the randomization strata.

Analysis of iDOR The iDOR is analyzed for responding participants (confirmed iCR or iPR). iDOR will be defined as the time from the date of first response to the date of iPD or death (any cause), whichever occurs first, and is analyzed using KM methods. Responding participants completing study follow-up or initiating a new anticancer therapy prior to iPD are censored at the last known date the participant was progression-free prior to completing follow-up or initiating the new therapy. The KM curve and median iDOR (and 95% CI) is summarized for each arm. Comparison of iDOR between the experimental arm and control arm, if tested, is based on the stratified log-rank test, stratified by the randomization strata. The iDOR HR (and 95% CI) is summarized based on the Cox proportional hazard model.

Analysis of Exploratory Efficacy Endpoints: The DSS, defined as, time from randomization to death resulting from NSCLC is analyzed in the same manner as OS. Endpoints of DCR, PFS, ORR, and DOR as measured using RECIST v1.1 is analyzed in the same manner as these endpoints using iRECIST.

Demographic and Baseline Characteristics: Demographic data and baseline characteristics including age, gender, race/ethnicity, ECOG performance status, screening height and weight, and limited medical history is summarized for each arm.

Analysis of Treatment Exposure: Exposure to study drug is summarized for each treatment arm.

Analysis of Safety” Overall safety is assessed for each treatment arm by descriptive analyses using tabulated frequencies of AEs by grade using CTCAE Version 5.0 in terms of treatment-emergent AEs, SAEs, and clinically significant changes in safety laboratory tests, and vital signs.

Analysis of Other Endpoints: Whole slide images and correlations with participant outcomes is explored. Tumor molecular profiling and blood analyses including assessment of ctDNA testing is explored.

Determination of Sample Size: The primary efficacy endpoint is OS. The study is planned for 462 participants to be randomized 2:1 to the experimental arm or control arm. A total of 462 participants are expected to accrue 357 deaths during the study with a 24-month enrollment period and 12-month follow-up period (after the last participant is enrolled). A total of 462 participants with 357 deaths have 90% power to detect a HR of 0.70, based on median OS of 8.3 as observed across multiple clinical trials in second line NSCLC (Camps 2006, Fossella 2002, Fossella 2000, Hanna 2004), (Paz-Ares 2008, Ramlau 2006) and 11.9 months for the control and experimental arms, respectively, with 5% type 1 error rate.

### REFERENCES

1. AACR. The IL15 Superagonist ALT-803 plus Nivolumab Has Antitumor Activity. Cancer Discovery. 2018; 8:OF16-OF.
2. Acheampong E, Allsopp R C, Page K, et al. Meta-Analysis of Circulating Tumor Cell PD-L1 Expression and the Association with Clinical Outcomes in Non-Small Cell Lung Cancer. Clin Chem. 2024; 70:234-49.
3. Ahmadzadeh M, Johnson L A, Heemskerk B, et al. Tumor antigen-specific CD8 T cells infiltrating the tumor express high levels of PD-1 and are functionally impaired. Blood. 2009; 114:1537-44.
4. American Cancer Society. American Cancer Society, Cancer Facts and Figures 2024. Atlanta: American Cancer Society; 2024. 2024.
5. Anagnostou V, Ho C, Nicholas G, et al. ctDNA response after pembrolizumab in non-small cell lung cancer: phase 2 adaptive trial results. Nature Medicine. 2023; 29:2559-69.
6. Borghaei H, Gettinger S, Vokes E E, et al. Five-Year Outcomes From the Randomized, Phase III Trials CheckMate 017 and 057: Nivolumab Versus Docetaxel in Previously Treated Non-Small-Cell Lung Cancer. J Clin Oncol. 2021; 39:723-33.
7. Borghaei H, Paz-Ares L, Horn L, et al. Nivolumab versus Docetaxel in Advanced Nonsquamous Non-Small-Cell Lung Cancer. N Engl J Med. 2015; 373:1627-39.
8. Brahmer J, Reckamp K L, Baas P, et al. Nivolumab versus Docetaxel in Advanced Squamous-Cell Non-Small-Cell Lung Cancer. N Engl J Med. 2015; 373:123-35.
9. Braud V M, Allan D S, O'Callaghan C A, et al. HLA-E binds to natural killer cell receptors CD94/NKG2A, B and C. Nature. 1998; 391:795-9.
10. Bucks C M, and Katsikis P D. New insights into classical costimulation of CD8+ T cell responses. Adv Exp Med Biol. 2009; 633:91-111.
11. Camps C, Massuti B, Jimenez A, et al. Randomized phase III study of 3-weekly versus weekly docetaxel in pretreated advanced non-small-cell lung cancer: a Spanish Lung Cancer Group trial. Ann Oncol. 2006; 17:467-72.
12. Christopher M J, Petti A A, Rettig M P, et al. Immune Escape of Relapsed AML Cells after Allogeneic Transplantation. N Engl J Med. 2018; 379:2330-41.
13. Dhatchinamoorthy K, Colbert J D, and Rock K L. Cancer Immune Evasion Through Loss of MHC Class I Antigen Presentation. Front Immunol. 2021; 12:636568.
14. Eisenhauer E A, Therasse P, Bogaerts J, et al. New response evaluation criteria in solid tumours: revised RECIST guideline (version 1.1). Eur J Cancer. 2009; 45:228-47.
15. Ellis-Connell A L, Balgeman A J, Kannal N M, et al. IL-15 Superagonist N-803 Enhances IFNgamma Production of MAIT Cells in SIV (+) Macaques. Infect Immun. 2022; 90:e0025922.
16. Felip E, Ardizzoni A, Ciuleanu T, et al. CheckMate 171: A phase 2 trial of nivolumab in patients with previously treated advanced squamous non-small cell lung cancer, including ECOG PS 2 and elderly populations. Eur J Cancer. 2020; 127:160-72.
17. Ferlay J, Ervik M, Lam F, et al. Global Cancer Observatory Cancer Today. 2024.
18. Foltz J A, Hess B T, Bachanova V, et al. Phase 1 trial of N-803, an IL-15 receptor agonist, with rituximab in patients with indolent non-Hodgkin lymphoma. Clinical Cancer Research. 2021; doi: 10.1158/1078-0432.CCR-20-4575:
19. Fossella F V. Docetaxel in second-line treatment of non-small-cell lung cancer. Clin Lung Cancer. 2002; 3 Suppl 2:S23-8.
20. Fossella F V, DeVore R, Kerr R N, et al. Randomized phase III trial of docetaxel versus vinorelbine or ifosfamide in patients with advanced non-small-cell lung cancer previously treated with platinum-containing chemotherapy regimens. The TAX 320 Non-Small Cell Lung Cancer Study Group. J Clin Oncol. 2000; 18:2354-62.
21. Garon E B, Ciuleanu T-E, Arrieta O, et al. Ramucirumab plus docetaxel versus placebo plus docetaxel for second-line treatment of stage IV non-small cell lung cancer after disease progression on platinum-based therapy (REVEL): a multicentre, double-blind, randomised phase 3 trial. Lancet. 2014; 384 (9944):665-673
22. Genova C, Dellepiane C, Carrega P, et al. Therapeutic Implications of Tumor Microenvironment in Lung Cancer: Focus on Immune Checkpoint Blockade. Front Immunol. 2021; 12:799455.
23. Han K P, Zhu X, Liu B, et al. IL-15:IL-15 receptor alpha superagonist complex: high-level coexpression in recombinant mammalian cells, purification and characterization. Cytokine. 2011; 56:804-10.
24. Hanna N, Shepherd F A, Fossella F V, et al. Randomized phase III trial of pemetrexed versus docetaxel in patients with non-small-cell lung cancer previously treated with chemotherapy. J Clin Oncol. 2004; 22:1589-97.
25. Herbst R S, Garon E B, Kim D W, et al. Five Year Survival Update From KEYNOTE-010: Pembrolizumab Versus Docetaxel for Previously Treated, Programmed Death-Ligand 1-Positive Advanced NSCLC. J Thorac Oncol. 2021; 16:1718-32.
26. Im S J, Hashimoto M, Gerner M Y, et al. Defining CD8+ T cells that provide the proliferative burst after PD-1 therapy. Nature. 2016; 537:417-21.
27. Jenkins R W, Barbie D A, and Flaherty K T. Mechanisms of resistance to immune checkpoint inhibitors. Br J Cancer. 2018; 118:9-16.
28. Kim P S, Kwilas A R, Xu W, et al. IL-15 superagonist/IL-15RalphaSushi-Fc fusion complex (IL-15SA/IL-15RalphaSu-Fc; ALT-803) markedly enhances specific subpopulations of NK and memory CD8+ T cells, and mediates potent anti-tumor activity against murine breast and colon carcinomas. Oncotarget. 2016; 7:16130-45.
29. Knetki-Wroblewska M, Kowalski D M, and Krzakowski M. Nivolumab for Previously Treated Patients with Non-Small-Cell Lung Cancer-Daily Practice versus Clinical Trials. J Clin Med. 2020; 9:
30. Knudson K M, Hicks K C, Alter S, et al. Mechanisms involved in IL-15 superagonist enhancement of anti-PD-L1 therapy. J Immunother Cancer. 2019; 7:82.
31. Li S, Manochakian R, Chen R, et al. Clinical outcomes of atezolizumab versus standard-of care docetaxel with and without ramucirumab in patients with advanced non-small-cell lung cancer who received prior immunotherapy. Front Oncol. 2024; 14:1306311.
32. Lo Monaco E, Tremante E, Cerboni C, et al. Human leukocyte antigen E contributes to protect tumor cells from lysis by natural killer cells. Neoplasia. 2011; 13:822-30.
33. Lui G, Minnar C M, Soon-Shiong P, et al. Exploiting an Interleukin-15 Heterodimeric Agonist (N803) for Effective Immunotherapy of Solid Malignancies. Cells. 2023; 12:34. Luo B, Cheung H W, Subramanian A, et al. Highly parallel identification of essential genes in cancer cells. Proc Natl Acad Sci USA. 2008; 105:20380-5.
35. Margolin K, Morishima C, Velcheti V, et al. Phase I Trial of ALT-803, A Novel Recombinant IL15 Complex, in Patients with Advanced Solid Tumors. Clin Cancer Res. 2018; 24:5552-61.
36. Mathios D, Park C K, Marcus W D, et al. Therapeutic administration of IL-15 superagonist complex ALT-803 leads to long-term survival and durable antitumor immune response in a murine glioblastoma model. Int J Cancer. 2016; 138:187-94.
37. National Comprehensive Cancer Network Inc. NCCN Guidelines Version 2.2024; Non-Small Cell Lung Cancer. 2024;
38. Neelapu S S, Tummala S, Kebriaei P, et al. Chimeric antigen receptor T-cell therapy-assessment and management of toxicities. Nat Rev Clin Oncol. 2018; 15:47-62.
39. Passaro A, Brahmer J, Antonia S, et al. Managing Resistance to Immune Checkpoint Inhibitors in Lung Cancer: Treatment and Novel Strategies. J Clin Oncol. 2022; 40:598-610.
40. Paz-Ares L, Ross H, O'Brien M, et al. Phase III trial comparing paclitaxel poliglumex vs docetaxel in the second-line treatment of non-small-cell lung cancer. Br J Cancer. 2008; 98:1608-13.
41. Ramlau R, Gervais R, Krzakowski M, et al. Phase III study comparing oral topotecan to intravenous docetaxel in patients with pretreated advanced non-small-cell lung cancer. J Clin Oncol. 2006; 24:2800-7.
42. Ren S, Feng J, Ma S, et al. KEYNOTE-033: Randomized phase 3 study of pembrolizumab vs docetaxel in previously treated, PD-L1-positive, advanced NSCLC. Int J Cancer. 2023; 153:623-34.
43. Roche P A, and Furuta K. The ins and outs of MHC class II-mediated antigen processing and presentation. Nat Rev Immunol. 2015; 15:203-16.
44. Romee R, Cooley S, Berrien-Elliott M M, et al. First-in-human phase 1 clinical study of the IL-15 superagonist complex ALT-803 to treat relapse after transplantation. Blood. 2018; 131:2515-27.
45. Sari G, and Rock K L. Tumor immune evasion through loss of MHC class-I antigen presentation. Curr Opin Immunol. 2023; 83:102329.
46. Seymour L, Bogaerts J, Perrone A, et al. iRECIST: guidelines for response criteria for use in trials testing immunotherapeutics. Lancet Oncol. 2017; 18:e143-e52.
47. Taxotere® [docetaxel] prescribing information. dailymed.nimh.nih.gov.
48. Wang J, Lu S, Yu X, et al. Tislelizumab Plus Chemotherapy vs Chemotherapy Alone as First-line Treatment for Advanced Squamous Non-Small-Cell Lung Cancer: A Phase 3 Randomized Clinical Trial. JAMA Oncol. May 1 2021; 7(5):709-717.
49. Wang Z, Zhao J, Ma Z, et al. Tislelizumab plus chemotherapy as first-line treatment for lung cancer in Chinese Patients [ESMO-IO Abstract 66P]. Ann Oncol. 2020; 31(7_suppl):S1441-51.
50. Wen M, Li Y, Qin X, et al. Insight into Cancer Immunity: MHCs, Immune Cells and Commensal Microbiota. Cells. 2023; 12:
51. Wherry E J, Blattman J N, Murali-Krishna K, et al. Viral persistence alters CD8 T-cell immunodominance and tissue distribution and results in distinct stages of functional impairment. J Virol. 2003; 77:4911-27.
52. Wong H C, Jeng E K, and Rhode P R. The IL-15-based superagonist ALT-803 promotes the antigen-independent conversion of memory CD8 (+) T cells into innate-like effector cells with antitumor activity. Oncoimmunology. 2013; 2:e26442.
53. Wrangle J, Velcheti V, Patel M, et al. A37 N-803 Plus Nivolumab for Advanced or Metastatic Non-Small Cell Lung Cancer: Update on Phase II Experience of Combination PD1 Blockade with an IL-15 Superagonist. Journal of Thoracic Oncology. 2020; 15:S24-S5.
54. Wrangle J M, Velcheti V, Patel M R, et al. ALT-803, an IL-15 Superagonist, in Combination with Nivolumab in Metastatic Non-Small Cell Lung Cancer, a non-randomized, open-label, phase 1b trial. Lancet Oncol. 2018; 19(5): 694-704.
55. Wu S, Wang L, Li W, et al. Comparison between the first-line and second-line immunotherapy drugs in the progression-free survival and overall survival in advanced non-small cell lung cancer: a systematic review and meta-analysis of randomized controlled trials. Ann Palliat Med. 2021; 10:1717-26.
56. Yang Y, and Lundqvist A. Immunomodulatory Effects of IL-2 and IL-15; Implications for Cancer Immunotherapy. Cancers (Basel). 2020; 12(12):3586.
57. Yue D, Wang W, Liu H, et al. LBA58 Pathological response to neoadjuvant tislelizumab (TIS) plus platinum-doublet (PtDb) chemotherapy (CT) in resectable stage II-IIIA NSCLC patients (pts) in the phase III (Ph3) RATIONALE-315 trial. Annals of Oncology. 2023; 34:S1299.
58. Zajac A J, Blattman J N, Murali-Krishna K, et al. Viral immune evasion due to persistence of activated T cells without effector function. J Exp Med. 1998; 188:2205-13.
59. Zhang Q, Luo J, Wu S, et al. Prognostic and Predictive Impact of Circulating Tumor DNA in Patients with Advanced Cancers Treated with Immune Checkpoint Blockade. Cancer Discov. 2020; 10:1842-53.
60. Zhang X, Beachler D C, Masters E, et al. Health care resource utilization and costs associated with advanced or metastatic nonsmall cell lung cancer in the United States. J Manag Care Spec Pharm. 2022; 28:255-65.
61. Zhou C, Huang D, Yu X, et al. Results from RATIONALE 303: A global phase 3 study of tislelizumab (TIS) vs docetaxel (TAX) as second- or third-line therapy for patients with locally advanced or metastatic NSCLC [abstract]. Cancer Res. 2021; 81(13_Suppl): Abstract nr CT039.
62. Zhou X, Ni Y, Liang X, et al. Mechanisms of tumor resistance to immune checkpoint blockade and combination strategies to overcome resistance. Front Immunol. 2022; 13:915094.
63. Zhu X, Marcus W D, Xu W, et al. Novel human interleukin-15 agonists. J Immunol. 2009; 183:3598-607.

1. A method for treating non-small cell lung carcinoma (NSCLC) in a human subject, wherein the subject has been previously treated with an immune checkpoint inhibitor and subsequently experienced disease progression, the method comprising administering to the subject:
a. an effective amount of an interleukin-15 (IL-15) protein or fusion protein derivative thereof, or an interleukin-15:interleukin-15 receptor subunit alpha (IL-15:IL-15Rα) protein or fusion protein derivative thereof;
b. an effective amount of a PD-1 inhibitor antibody; and
c. an effective amount of a taxane.

2. The method of claim 1, wherein the NSCLC is advanced or metastatic NSCLC.

3. The method of claim 1, wherein the subject has an actionable genomic alteration (AGA).

4. The method of claim 1, wherein the PD-1 inhibitor antibody is an IgG4 antibody.

5. The method of claim 1, wherein IL-15:IL-15Rα fusion protein derivative is an IL-15N72D:IL-15RαSu/Fc complex (N-803), wherein the N-803 comprises a dimeric IL-15RαSu/Fc and two IL-15N72D molecules.

6. The method of claim 1, wherein the PD-1 inhibitor antibody is selected from the group consisting of tislelizumab, nivolumab, pembrolizumab, MEDI0680 and cemiplimab.

7. The method of claim 6, wherein the PD-1 inhibitor antibody is tislelizumab.

8. The method of claim 1, wherein the taxane is docetaxel.

9. The method of claim 1, wherein the IL-15 protein or fusion protein derivative thereof, or the IL-15:IL-15Rα protein or fusion protein derivative thereof, retains binding affinity and agonist activity to interleukin-15 receptor subunits beta gamma (IL-15Rβγ).

10. The method of claim 1, wherein the effective amount of the IL-15 protein or fusion protein derivative thereof, or the IL-15:IL-15Rα protein or fusion protein derivative thereof, is administered subcutaneously to the subject; and the effective amount of the PD-1 inhibitor antibody and the effective amount of the taxane are each administered intravenously to the subject.

11. The method of claim 1, wherein the IL-15 protein or fusion protein derivative thereof, or the IL-15:IL-15Rα protein or fusion protein thereof is in a pharmaceutical composition; the PD-1 inhibitor antibody is in a pharmaceutical composition; and the taxane is in a pharmaceutical composition.

12. The method of claim 1, wherein the subject is initially administered a first cycle of treatment comprising administering the IL-15 protein or fusion protein derivative thereof, or the IL-15:IL-15Rα protein or fusion protein derivative thereof, subcutaneously to the subject, followed by administering the PD-1 inhibitor antibody intravenously to the subject, followed by administering the taxane intravenously to the subject, followed by a second cycle of treatment comprising further administering the IL-15 protein or fusion protein derivative thereof, or the IL-15:IL-15Rα protein or fusion protein derivative thereof subcutaneously to the subject, followed by administering the PD-1 inhibitor antibody intravenously to the subject, followed by administering the taxane intravenously to the subject 19 to 23 days after completion of the first cycle.

13. The method of claim 12, wherein the subject is administered a third cycle of treatment comprising further administering the IL-15 protein or fusion protein derivative thereof, or the IL-15:IL-15Rα protein or fusion protein derivative thereof, subcutaneously to the subject, followed by administering the PD-1 inhibitor antibody intravenously to the subject 40 to 44 days after the completion of the first cycle.

14. A kit for use in treating NSCLC in a subject, the kit comprising:
a. a first vile comprising a composition comprising an IL-15 protein or fusion protein derivative thereof, or an IL-15:IL-15Rα protein or fusion protein derivative thereof, wherein the composition is formulated for subcutaneous administration to the subject;
b. a second vile comprising a composition comprising a PD-1 inhibitor antibody, wherein the composition is formulated for intravenous administration to the subject;
c. a third vile comprising a composition comprising a taxane, wherein the composition is formulated for intravenous administration to the subject; and
d. instructions for the use thereof.

15. The kit claim 14, wherein the IL-15:IL-15Rα fusion protein derivative is N-803.

16. The kit of claim 14, wherein the PD-1 antibody is tislelizumab.

17. The kit of claim 14, wherein the taxane is docetaxel.


SEQUENCE LISTING (1 sequence)

SEQ ID NO: 1 - protein, 65 aa, Homo sapiens
ITCPPPMSVEHADIWVKSYSLYSRERYICNSGFKRKAGTSSLTECVLNKATNVAHWTTPSLKCIR